SEQUENCE LISTING (4 sequences)

SEQ ID NO: 1 - protein, 15 aa, Homo sapiens
KVEKIGEGTYGVVYK

SEQ ID NO: 2 - protein, 13 aa, Homo sapiens
KKSRGDYMTMQIG

SEQ ID NO: 3 - protein, 16 aa, Homo sapiens
GGMEDIYFEFMGGKKK

SEQ ID NO: 4 - protein, 13 aa, Homo sapiens
KKKGQEEEYVFIE

(19) United States
(12) Patent Application Publication
(10) Pub. No.: US 2023/0339889 A1
(43) Pub. Date: Oct. 26, 2023

(54) DERIVATIVES OF 2-OXO-N-(4-(PYRIMIDIN-4-YLOXY/THIO)PHENYL)-1,2-DIHYDROPYRIDINE-3-CARBOXAMIDE FOR USE AS PROTEIN KINASE INHIBITORS FOR THERAPY

(71) Applicant: AUCENTRA THERAPEUTICS PTY LTD, Adelaide (AU)

(72) Inventors: Shudong WANG, Adelaide (AU); Aik Wye GOH, Thebarton (AU)

(21) Appl. No.: 18/005,004

(22) PCT Filed: Jul. 9, 2021
(86) PCT No.: PCT/AU2021/050737

§ 371 Date: Jan. 10, 2023

(30) Foreign Application Priority Data
Jul. 10, 2020 (AU) 2020902392

(51) Int. Cl. C07D 401/12

(52) U.S. Cl. CPC C07D 401/12

(57) ABSTRACT

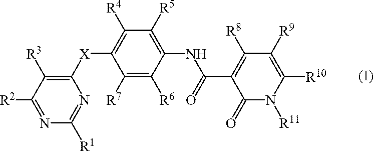

2-oxo-N-(4-(pyrimidin-4-yloxy/thio)phenyl)-1,2-dihydropyridine-3-carboxamide derivatives are disclosed for use in the prevention and/or treatment of proliferative cell diseases and conditions including cancers. The compounds are considered to be capable of inhibiting cell proliferation and cause cancer cell apoptosis by inhibiting the activity of receptor tyrosine kinases (RTKs) such as TYRO3, AXL, MER and/or MET. The compounds have the general structure (I) shown below:

### TECHNICAL FIELD

The present disclosure relates to a novel class of inhibitors of protein kinases useful in the treatment of proliferative cell diseases and conditions including cancers.

### PRIORITY DOCUMENT

The present application claims priority from Australian Provisional Patent Application No 2020902392 titled “Protein kinase inhibitors for therapy” and filed on 10 Jul. 2020, the content of which is hereby incorporated by reference in its entirety.

### BACKGROUND

There is an ongoing need to identify and develop new compounds for treating proliferative diseases and conditions including cancers. Among the numerous “targets” for potential anti-proliferative compounds under investigation are the group of enzymes known as receptor tyrosine kinases (RTKs). RTKs are cell surface proteins that transmit signals from the extracellular environment to the cell cytoplasm and nucleus to regulate cellular events such as survival, growth, proliferation, differentiation, adhesion and migration. Impaired gene functions by mutations or deletions may cause the abnormal expression of protein kinases, which, in turn, entails tumour formation and progression.

The TAM subfamily consists of three RTKs, namely TYRO3, AXL and MER (Graham et al., Nat Rev Cancer 14:769-785, 2014; Linger et al. Adv Cancer Res 100:35-83, 2008). TAM kinases are characterised by an extracellular ligand binding domain consisting of two immunoglobulin-like domains and two fibronectin type III domains. Two ligands, Growth Arrest Specific 6 (GAS6) and Protein S (PROS1), have been identified for TAM kinases. GAS6 can bind to and activate all three TAM kinases, while PROS1 is a ligand for MER and TYRO3 (Graham et al., supra). The activation of TAM receptors results in signalling down several growth promoting pathways, such as the PI3K/AKT, MAPK, and PKC pathways. Moreover, TAM receptors are essential regulators of epithelial-mesenchymal transition (EMT), which causes therapeutic resistance, metastasis and immune cell suppression, suggesting important roles for TAM in cancer biology and therapies.

AXL (also known as UFO, ARK, JTK11 and TYRO7) was originally identified as a transforming gene from DNA of patients with chronic myelogenous leukaemia (O′Bryan et al., Mol Cell Biol 11:5016-5031, 1991; Graham et al., supra). The GAS6 binds to AXL and subsequently auto-phosphorylates and activates AXL kinase (Stitt TN et al., Cell 80(4):661-670, 1995; Li et al., Oncogene 1;28(39):3442-3455, 2009). AXL activates several downstream signalling pathways including PI3K/AKT, Raf/MAPK, PKC (Feneyrolles et al., Mol Cancer Ther 13:2141-2148, 2014). AXL overexpression has been detected in a majority of human cancers, including acute myeloid leukaemia (Hong C-C et al., Cancer Lett 268(2):314-324, 2008), breast cancer (Berclaz G et al., Ann Oncol 12(6):819-824, 2001; Zhang YX et al., Cancer Res 68(6):1905-1915, 2008; Gjerdrum C et al., Proc Natl Acad Sci U S A 107(3):1124-1129, 2010), gastric (Wu CW et al., Anticancer Res 22(2B):1071-1078, 2002) and lung cancer (Shieh YS et al., Neoplasia 7(12):1058-1064, 2005), melanoma (Quong RY et al., Melanoma Res 4(5):313-319, 1994), osteosarcoma (Han J et al., Biochem Biophys Res Commun 435(3):493-500, 2013), renal cell carcinoma (Gustafsson A et al., Clin Cancer Res 15(14):4742-4749, 2009), etc. More recently, the AXL receptor has been found to mediate resistance to several different cancer therapies, including chemotherapy, radiation, and inhibitors of EGFR and PI3K. Therefore, targeting AXL might be a promising strategy for the treatment of various malignant tumours.

MER kinase (also known as MERTK, EYK, RYK, RP38, NYK and TYRO12) was originally identified as a phospho-protein from a lymphoblastoid expression library (Graham et al., Oncogene 10:2349-2359, 1995). Both GAS6 and PROS1 can bind to MER and induce the phosphorylation and activation of MER kinase. Like AXL, MER activation also conveys downstream signalling pathways including PI3K/AKT and Raf/MAPK. Aberrant expression of MER in various malignant tumours, such as melanoma (Schlegel et al., J Clin Invest 123(5):2257-2267, 2013), gastric cancer (Yi et al., Oncotarget 8(57):96656-96667, 2017), leukaemia (Linger et al., Blood 122(9):1599-1609, 2013; Lee-Sherick et al., Oncogene 32(46):5359-5368, 2013), and lung cancer (Xie et al., Oncotarget 6(11):9206-9219, 2015), plays a pivotal role in the process of oncogenesis.

TYRO3 (also known as DTK, SKY, RSE, BRT, TIF, ETK2) was originally identified through a PCR-based cloning study (Lai et al., Neuron 6:691-670, 1991). Both ligands, GAS6 and PROS1, can bind to and activate TYRO3. TYRO3 appears to have a critical role in immunity, phagocytosis, haemostasis and neuronal disease. TYRO3 and ligand overexpression have been shown in a wide range of cancers, and correlate with poor prognosis in a variety of tumour types. Through AKT/NFκB signalling, TYRO3 exerts pro-survival effects and promotes cancer cell growth (Crosier et al., Leuk Lymphoma 18:443-449, 1995). Protein levels of TYRO3 and AXL are undetectable in normal thyroid cells but are significantly upregulated and activated in thyroid cancer cells (Avilla et al., Cancer Res 71:1792-1804, 2011). Activated TYRO3 promotes the survival, invasion, migration, proliferation and transformation of cancer cells. TYRO3 was also shown to promote chemoresistance in breast cancer (Ekyalongo et al., Anticancer Res 34:3337-3345, 2014), and ovarian cancer (Lee et al., Mol Med Rep 12:1485-1492, 2015). TYRO3 promotes phagocytosis and inhibits inflammation, allowing resistance to antitumor treatments to further cancer progression (Liu et al., J Immunother 35:299-308, 2012). Taken together, the studies suggest that inhibition of TYRO3 and its signalling pathways could have therapeutic benefits in cancer treatment.

TAM inhibition not only has direct activity against neoplastic cells, but also activates the anticancer immune response (Akalu YT et al. Immunol Rev 276(1):165-177, 2017), thus, TAM inhibitors represent an attractive approach for the treatment of cancer. In addition, TAM inhibitors may be combined with other targeted therapies, chemotherapies, radiation, or immunotherapeutic agents to achieve maximal efficacy in the clinic (Yokoyama et al., Cancer Res 79:1996-2008, 2019).

MET, also known as the N-methyl-N′-nitroso-guanidine human osteosarcoma transforming gene, is a proto-oncogene encoding a receptor tyrosine kinase c-MET for hepatocyte growth factor (HGF) (Bladt et al., Nature 376:768-771, 1995; Sattler et al., Curr Oncol Rep 102-108, 2007). The binding of HGF results in c-MET dimerisation and autophosphorylation, which in turn activates the MAPK, PI3K, SRC and STAT signalling pathways (Ma et al., Cancer Metastasis Rev 309-325, 2003). Aberrant MET expression is widely observed in various malignancies, particularly non-small cell lung cancer, gastrointestinal cancer, and hepatocellular carcinoma (Ichimura et al., Jpn J Cancer Res 87:1063-1069, 1996; Siegfried et al., Ann Thorac Surg 66:1915-1918, 1998; Goyal et al., Clin Cancer Res 19:2310-2318, 2013; Hack et al., Oncotarget 5:2866-2880, 2014). Therefore, MET has become an attractive target for cancer treatment and drug development.

The present applicant has now identified a new class of compounds for use in the prevention and/or treatment of proliferative diseases and conditions including cancers. While not wishing to be bound by theory, it is considered that these novel compounds are capable of inhibiting cell proliferation, therapeutic resistance, metastasis, and immune cell suppression, by inhibiting the activity of one or more protein kinases such as RTKs, and especially one or more of TAM and/or MET family protein kinases, and/or their mutant forms.

### SUMMARY

The present invention is directed to a compound of Formula I:

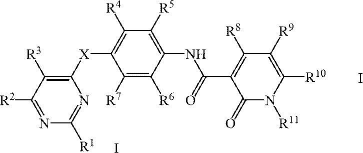

wherein:
X is O or S;
R¹, R², R³, R⁴, R⁵, R⁶, R⁷, R⁸, R⁹ and R¹⁰ are each independently selected from the group consisting of H, alkyl, alkyl-R¹², aryl, aryl-R¹², aralkyl, aralkyl-R¹², alicyclic, heterocyclic, halogen, NO₂, CN, CF₃, O—CF₃, OH, O-alkyl, COR¹², COOR¹², O-aryl, O-R¹², amino, NH-alkyl, NH-aryl, N-(alkyl)₂, N-(aryl)₂, N-(alkyl)(aryl), NH-R¹², NH-alkyl-N(alkyl)₂, N-(R¹²)(R¹³), N-(alkyl)(R¹²), N-(aryl)(R¹²), COOH, CONH₂, CONH-alkyl, CONH-aryl, CONH-alicyclic, CON-(alkyl)(R¹²), CON(aryl)(R¹²), CONH-R¹², CON-(R¹²)(R¹³), S-alkyl, SO₃H, SO₂-alkyl, SO₂-alkyl-R¹², SO₂-aryl, SO₂-aryl-R¹², SO₂NH₂, SO₂NH—R¹², SO₂N—(R¹²)(R¹³), CO-alkyl, CO-alkyl-R¹², CO-aryl, and CO-aryl-R¹², wherein said alkyl, aryl, aralkyl, alicyclic and heterocyclic groups may be optionally substituted with one or more groups selected from C₁₋₆ alkyl, O-C₁₋₆ alkyl, CN, OH, NH₂, COOH, CONH₂, CF₃, OCF₃ and halogen;
wherein R¹² and R¹³ are independently selected from COOH, SO₃H, OSO₃H, SONHCH₃, SONHCH₂CH₃, SO₂CH₃, SO₂CH₂CH₃, PO₃H₂ and OPO₃H₂, mono-, di- and poly-hydroxylated alicyclic groups, di- or poly-hydroxylated aliphatic or aryl groups, and N-, O- and/or S-containing heterocyclic groups optionally substituted with one or more C₁₋₆ alkyl, hydroxyl, carbonyl, amino or alkoxy groups, wherein the N-, O-and/or S-containing heterocyclic groups may optionally be linked to the rest of the compound through an alkyl, amine, alkoxy or ketone bridge, wherein at least two of R¹, R² and R³ are other than H; and
R¹¹ is selected from phenyl-R¹⁴,
wherein R¹⁴ is selected from C₁₋₆ alkyl, O-C₁₋₆ alkyl, CN, OH, NH₂, COOH, CONH₂, CF₃, OCF₃ and halogen;
or a pharmaceutically acceptable salt, solvate or prodrug thereof.

In a second aspect, the present disclosure provides the use of a compound as defined in the first aspect or a pharmaceutically acceptable salt, solvate or prodrug thereof, for treating cancer or another proliferative cell disease or condition.

In a third aspect, the present disclosure provides a method of treating cancer or another proliferative cell disease or condition in a subject, the method comprising administering to said subject a therapeutically effective amount of a compound as defined in the first aspect or a pharmaceutically acceptable salt, solvate or prodrug thereof, optionally in combination with a pharmaceutically acceptable carrier, diluent and/or excipient.

In a fourth aspect, the present disclosure provides the use of a compound as defined in the first aspect, or a pharmaceutically acceptable salt, solvate or prodrug thereof, in the manufacture of a medicament for treating cancer or another proliferative cell disease or condition.

In a fifth aspect, the present disclosure provides a pharmaceutical composition or medicament comprising a compound as defined in the first aspect, or a pharmaceutically acceptable salt, solvate or prodrug thereof, and a pharmaceutically acceptable carrier, diluent and/or excipient.

In a sixth aspect, the present disclosure provides a method for modulating protein kinase activity in a cell, comprising introducing to or contacting said cell with an effective amount of a compound as defined in the first aspect or a pharmaceutically acceptable salt, solvate or prodrug thereof.

### DETAILED DESCRIPTION

The present applicant has now identified a new class of pyrimidin-2-amine derivatives, particularly derivatives of 2-oxo-N-(4-(pyrimidin-4-yloxy/thio)phenyl)-1,2-dihydropyridine-3-carboxamide, suitable for use in the prevention and/or treatment of proliferative cell diseases and conditions including cancers, which possess desirable biological activity (eg the compounds may inhibit cell proliferation and cause cancer cell apoptosis by inhibiting the activity of receptor tyrosine kinases (RTKs) such as TYRO3, AXL, MER and/or MET).

According to a first aspect, the present disclosure provides a compound of Formula I shown below:

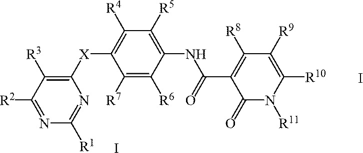

wherein:
X is O or S;
R¹, R², R³, R⁴, R⁵, R⁶, R⁷, R⁸, R⁹ and R¹⁰ are each independently selected from the group consisting of H, alkyl, alkyl-R¹², aryl, aryl-R¹², aralkyl, aralkyl-R¹², alicyclic, heterocyclic, halogen, NO₂, CN, CF₃, O—CF₃, OH, O-alkyl, COR¹², COOR¹², O-aryl, O-R¹², amino, NH-alkyl, NH-aryl, N-(alkyl)₂, N-(aryl)₂, N-(alkyl)(aryl), NH-R¹², NH-alkyl-N(alkyl)₂, N-(R¹²)(R¹³), N-(alkyl)(R¹²), N-(aryl)(R¹²), COOH, CONH₂, CONH-alkyl, CONH-aryl, CONH-alicyclic, CON-(alkyl)(R¹²), CON(aryl)(R¹²), CONH-R¹², CON-(R¹²)(R¹³), S-alkyl, SO₃H, SO₂-alkyl, SO₂-alkyl-R¹², SO₂-aryl, SO₂-aryl-R¹², SO₂NH₂, SO₂NH—R¹², SO₂N—(R¹²)(R¹³), CO-alkyl, CO-alkyl-R¹², CO-aryl, and CO-aryl-R¹², wherein said alkyl, aryl, aralkyl, alicyclic and heterocyclic groups may be optionally substituted with one or more groups selected from C₁₋₆ alkyl, O-C₁₋₆ alkyl, CN, OH, NH₂, COOH, CONH₂, CF₃, OCF₃ and halogen;
wherein R¹² and R¹³ are independently selected from COOH, SO₃H, OSO₃H, SONHCH₃, SONHCH₂CH₃, SO₂CH₃, SO₂CH₂CH₃, PO₃H₂ and OPO₃H₂, mono-, di- and poly-hydroxylated alicyclic groups, di- or poly-hydroxylated aliphatic or aryl groups, and N-, O- and/or S-containing heterocyclic groups optionally substituted with one or more C₁₋₆ alkyl, hydroxyl, carbonyl, amino or alkoxy groups, wherein the N-, O-and/or S-containing heterocyclic groups may optionally be linked to the rest of the compound through an alkyl, amine, alkoxy or ketone bridge, wherein at least two of R¹, R² and R³ are other than H; and
R¹¹ is selected from phenyl-R¹⁴,
wherein R¹⁴ is selected from C₁₋₆ alkyl, O-C₁₋₆ alkyl, CN, OH, NH₂, COOH, CONH₂, CF₃, OCF₃ and halogen;
or a pharmaceutically acceptable salt, solvate or prodrug thereof.

In some embodiments, where present, the R¹² and/or R¹³ group(s) may provide the compound of Formula I with at least one water solubilising group. The presence of at least one water solubilising group may enhance in vivo absorption and oral bioavailability.

In some embodiments, where present, the R¹² and/or R¹³ group(s) comprise an N-, O- and/or S-containing heterocyclic group(s) (optionally substituted with one or more hydroxyl, amino or alkoxy groups) which may be linked to the rest of the compound by an alkyl bridge (eg a —CH₂— or —CH₂CH₂—bridge), amine bridge (eg —NH—, —NH—CH₂— and —NH—CH₂CH₂—), alkoxy bridge (eg —O—CH₂— and —O—CH₂CH₂—) or ketone bridge (eg a —C(═O)— bridge). For example, where the compound comprises an NH-R¹² group, R¹² may be an N-, O- and/or S-containing heterocyclic group (optionally substituted with one or more hydroxyl, amino or alkoxy groups) linked to the rest of the compound by, for example, a —CH₂—or —CH₂CH₂— alkyl bridge.

The compounds of Formula I have been found to possess anti-proliferative activity and are therefore considered to be of use in the treatment of proliferative cell diseases and conditions such as cancer, leukaemia, lymphoma and other diseases and conditions associated with uncontrolled cell proliferation (or, in other words, requires control of the cell cycle) such as, for example, some cardiovascular diseases or conditions such as restenosis and cardiomyopathy, some auto-immune diseases such as glomerulonephritis and rheumatoid arthritis, dermatological conditions such as psoriasis, and fungal or parasitic disorders. As used herein, an anti-proliferative effect within the scope of the present disclosure may be demonstrated by the ability to inhibit cell proliferation in an in vitro whole cell assay. An example(s) of such an assay, including methods for performance, are described in more detail in the Example 2 provided hereinafter.

In a second aspect, the present disclosure provides the use of a compound as defined in the first aspect or a pharmaceutically acceptable salt, solvate or prodrug thereof, for treating cancer or another proliferative cell disease or condition.

In a third aspect, the present disclosure provides a method of treating cancer or another proliferative cell disease or condition in a subject, the method comprising administering to said subject a therapeutically effective amount of a compound as defined in the first aspect or a pharmaceutically acceptable salt, solvate or prodrug thereof, optionally in combination with a pharmaceutically acceptable carrier, diluent and/or excipient.

In a fourth aspect, the present disclosure provides the use of a compound as defined in the first aspect, or a pharmaceutically acceptable salt, solvate or prodrug thereof, in the manufacture of a medicament for treating cancer or another proliferative cell disease or condition.

In a fifth aspect, the present disclosure provides a pharmaceutical composition or medicament comprising a compound as defined in the first aspect, or a pharmaceutically acceptable salt, solvate or prodrug thereof, and a pharmaceutically acceptable carrier, diluent and/or excipient.

In a sixth aspect, the present disclosure provides a method for modulating protein kinase activity in a cell, comprising introducing to or contacting said cell with an effective amount of a compound as defined in the first aspect or a pharmaceutically acceptable salt, solvate or prodrug thereof.

Preferably, the method of the sixth aspect modulates the activity of one or more protein kinases selected from RTKs, and especially one or more of TAM and/or MET family protein kinases.

In this specification, a number of terms are used which are well known to those skilled in the art. Nevertheless, for the purposes of clarity, a number of these terms are hereinafter defined.

As used herein, the term “treating” includes prophylaxis as well as the alleviation of established symptoms of a condition. As such, the act of “treating” a disease or condition therefore includes: (1) preventing or delaying the appearance of clinical symptoms of the disease or condition developing in a subject afflicted with or predisposed to the disease or condition; (2) inhibiting the disease or condition (ie arresting, reducing or delaying the development of the disease or condition or a relapse thereof (in case of a maintenance treatment) or at least one clinical or subclinical symptom thereof; and (3) relieving or attenuating the disease or condition (ie causing regression of the disease or condition or at least one of its clinical or subclinical symptoms).

As used herein, the term “alkyl” includes both straight chain and branched alkyl groups having from 1 to 8 carbon atoms (eg methyl, ethyl, propyl, isopropyl, butyl, isobutyl, tert-butyl, pentyl, hexyl, cyclopropyl, cyclobutyl, cyclopentyl etc).

As used herein, the term “aryl” refers to a substituted (mono- or poly-) or unsubstituted monoaromatic or polyaromatic group, wherein said polyaromatic group may be fused or unfused. The term therefore includes groups having from 6 to 10 carbon atoms (eg phenyl, naphthyl etc). It is also to be understood that the term “aryl” is synonymous with the term “aromatic”.

As used herein, the term “aralkyl” is used as a conjunction of the terms alkyl and aryl as defined above.

The term “aliphatic” takes its normal meaning in the art and includes non-aromatic groups such as alkanes, alkenes and alkynes and substituted derivatives thereof. The term includes groups having from 1 to 8 carbon atoms.

As used herein, the term “alicyclic” refers to a cyclic aliphatic group.

The term “halogen” refers to fluoro, chloro, bromo and iodo.

As used herein, the term “heterocyclic” refers to a saturated or unsaturated cyclic group comprising one or more heteroatoms in a ring system (eg a system comprising one or more rings (mono-or poly-), and wherein where more than one ring is present, the rings may be fused and/or unfused. As such, the term covers saturated heterocyclic groups such as a pyrrolidinyl, morpholinyl, aziridine and piperazine, and unsaturated heterocyclic groups (“heteroaryl” groups such as 2-pyridyl, 3-pyridyl, 4-pyridyl, 4-pyrimidyl, 5-indolyl, furan, thiophene and thiazole etc); and wherein at least one ring of the ring system contains from one to four heteroatoms selected from N, O and S as ring members (ie it contains at least one heterocyclic ring), and wherein the nitrogen and sulfur atoms can be oxidised and the nitrogen atom(s) can be quaternised. A heterocyclic group can be attached to the remainder of the molecule through an annular carbon or annular heteroatom, and it can be attached through any ring of the ring system, if that ring system is, for example a poly-ring system such as a bicyclic, tricyclic or fused ring system.

The term “derivative” as used herein, includes any chemical modification of an entity. Illustrative of such chemical modifications is the replacement of hydrogen by a halogen group, an alkyl group, an acyl group or an amino group.

As used herein, the phrase “manufacture of a medicament” includes the use of one or more of the compounds of Formula I directly as the medicament or in any stage of the manufacture of a medicament comprising one or more of the compounds of Formula I.

Some of the compounds of Formula I may exist as single stereoisomers, racemates, and/or mixtures of enantiomers and /or diastereomers. All such single stereoisomers, racemates and mixtures thereof, are encompassed within the scope of the present disclosure. The isomeric forms such as diastereomers, enantiomers, and geometrical isomers can be separated by physical and/or chemical methods known to those skilled in the art.

The term “pharmaceutically acceptable salt” as used herein, refers to salts that retain the desired biological activity of the compounds of Formula I, and include pharmaceutically acceptable acid addition salts and base addition salts. Suitable pharmaceutically acceptable acid addition salts of the compounds of Formula I may be prepared from an inorganic acid or from an organic acid. Examples of such inorganic acids are hydrochloric, sulfuric and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic. Additional information on pharmaceutically acceptable salts can be found in Remington’s Pharmaceutical Sciences, 19th Edition, Mack Publishing Co., Easton, PA 1995.

The term “solvate” refers to any form of the compounds of formula I, resulting from solvation of with an appropriate solvent. Such a form may be, for example, a crystalline solvate or a complex that maybe formed between the solvent and the dissolved compound.

The term “prodrug” means a compound that undergoes conversion to a compound of Formula I within a biological system, usually by metabolic means (eg by hydrolysis, reduction or oxidation). For example, an ester prodrug of a compound of Formula I containing a hydroxyl group may be convertible by hydrolysis in vivo to the compound of Formula I. Suitable esters of the compounds of Formula I containing a hydroxyl group may be, for example, acetates, citrates, lactates, tartrates, malonates, oxalates, salicylates, propionates, succinates, fumarates, maleates, methylene-bis-P-hydroxynaphthoates, gestisates, isethionates, di-p-toluoyltartrates, methanesulfonates, ethanesulfonates, benzenesulfonates, p-toluenesulfonates, cyclohexylsulfamates and quinates. As another example, an ester prodrug of a compound of Formula I containing a carboxy group may be convertible by hydrolysis in vivo to the compound of Formula I. Examples of ester prodrugs include those described by Leinweber FJ, Drug Metab Rev 18:379-439 (1987). Similarly, an acyl prodrug of a compound of Formula I containing an amino group may be convertible by hydrolysis in vivo to the compound of Formula I. Examples of prodrugs for these and other functional groups, including amines, are provided in Prodrugs: challenges and rewards, Valentino J Stella (ed), Springer, 2007.

In the case of compounds of Formula I that are solid, it will be understood by those skilled in the art that the compounds (or pharmaceutically acceptable salts, solvates or prodrugs thereof) may exist in different crystalline or polymorphic forms, all of which are encompassed within the scope of the present disclosure.

The term “therapeutically effective amount” or “effective amount” is an amount sufficient to effect beneficial or desired clinical results. A therapeutically effective amount can be administered in one or more administrations. Typically, a therapeutically effective amount is sufficient for treating a disease or condition or otherwise to palliate, ameliorate, stabilise, reverse, slow or delay the progression of a disease or condition such as, for example, cancer or another proliferative cell disease or condition. By way of example only, a therapeutically effective amount of a compound of Formula I, or a pharmaceutically acceptable salt, solvate or prodrug thereof, may comprise between about 0.1 and about 250 mg/kg body weight per day, more preferably between about 0.1 and about 100 mg/kg body weight per day and, still more preferably between about 0.1 and about 25 mg/kg body weight per day. However, notwithstanding the above, it will be understood by those skilled in the art that the therapeutically effective amount may vary and depend upon a variety of factors including the activity of the particular compound (or salt, solvate or prodrug thereof), the metabolic stability and length of action of the particular compound (or salt, solvate or prodrug thereof), the age, body weight, sex, health, route and time of administration, rate of excretion of the particular compound (or salt, solvate or prodrug thereof), and the severity of, for example, the cancer or other proliferative cell disease or condition to be treated.

The compounds of Formula I, and pharmaceutically acceptable salts, solvates and prodrugs thereof, are capable of inhibiting protein kinases, especially RTKs and may show higher selectivity for (ie to inhibit) TYRO3, AXL, MER and/or MET over other protein kinases. As such, the compounds of Formula I, and pharmaceutically acceptable salts, solvates and prodrugs thereof, which are believed to inhibit at least TYRO3, AXL, MER and/or MET, have utility in both in vitro and in vivo applications (eg in vitro cell-based assays) and as the basis of a therapeutic method of treating cancer or another proliferative cell disease or condition in a subject.

The compounds of Formula I may bear at least one water solubilising group (eg provided by R¹², and/or R¹³). The term “water solubilising group” will be well understood by those skilled in the art as referring to any polar functional group which either ionises or is capable of forming hydrogen bonds with water molecules to increase the water solubility of the compound (ie relative to the water solubility of the corresponding compound lacking the water solubilising group). Examples of suitable water solubilising groups and methods and considerations for their introduction are described in, for example, Fundamentals of Medicinal Chemistry by Gareth Thomas (publisher: John Wiley & Sons).

At least two of R¹, R² and R³ are other than H; such that the compound of formula (I) comprises a di- or tri-substituted pyrimidine group.

In some embodiments, R¹, R² and R³ are independently selected from the group consisting of H, alkyl (eg a C₁₋₆ alkyl or, preferably, a C₁₋₃ alkyl such as methyl, ethyl and C(CH₃)₂), CN, CF₃, amino (eg NH₂), O-alkyl (eg a O-C₁₋₃ alkyl such as O—CH₃), NH-alkyl (eg a NH-C₁₋₆ alkyl such as NH(C₅H₉) (ie NH-cyclopentyl) or, preferably, a NH-C₁₋₃ alkyl such as NH—CH₃), S-alkyl (eg a S-C₁₋₆ alkyl or, preferably, a S-C₁₋₃ alkyl such as S—CH₃ and S—CH(CH₃)₂, and halogen (preferably F, Br or Cl).

Preferably, R¹ is H, C₁₋₃ alkyl such as methyl, or amino (eg NH₂).

Preferably, R² is H, C₁₋₃ alkyl such as methyl, or amino (eg NH₂).

Preferably, R³ is H, C₁₋₃ alkyl such as methyl, O-alkyl (eg O—CH₃) or halogen (preferably, F or Cl).

In some embodiments, R⁴, R⁵, R⁶ and R⁷ are independently selected from the group consisting of H, alkyl (eg a C₁₋₆ alkyl or, preferably, a C₁₋₃ alkyl such as methyl, ethyl and C(CH₃)₂), CN, CF₃, amino (eg NH₂), O-alkyl (eg a O-C₁₋₃ alkyl such as O—CH₃), NH-alkyl (eg a NH-C₁₋₆ alkyl such as NH(C₅H₉) (ie NH-cyclopentyl) or, preferably, a NH-C₁₋₃ alkyl such as NH—CH₃), S-alkyl (eg a S-C₁₋₆ alkyl or, preferably, a S-C₁₋₃ alkyl such as S—CH₃ and SCH(CH₃)₂, and halogen (preferably F, Br or Cl).

Preferably, R⁴, R⁵, R⁶ and R⁷ are independently selected from H and halogen (preferably, F). Also, preferably, at least one of R⁴, R⁵, R⁶ and R⁷ is H.

In some preferred embodiments, one or two of R⁴, R⁵, R⁶ and R⁷ are halogen (preferably, F)

In some other preferred embodiments, R⁴, R⁵, R⁶ and R⁷ are all H.

In some embodiments, R⁸, R⁹ and R¹⁰ are independently selected from the group consisting of H, alkyl (eg a C₁₋₆ alkyl or, preferably, a C₁₋₃ alkyl such as methyl, ethyl and C(CH₃)₂, CN, CF₃, amino (eg NH₂), O-alkyl (eg a O-C₁₋₃ alkyl such as O—CH₂CH₃), NH-alkyl (eg a NH-C₁₋₆ alkyl such as NH(C₅H₉) (ie NH-cyclopentyl) or, preferably, a NH-C₁₋₃ alkyl such as NH—CH₃), S-alkyl (eg a S-C₁₋₆ alkyl or, preferably, a S-C₁₋₃ alkyl such as S—CH₃ and S—CH(CH₃)₂, and halogen (preferably F, Br or Cl).

Preferably, R⁸ is H, C₁₋₃ alkyl such as methyl, or O-C₁₋₃ alkyl such as O—CH₂CH₃.

Preferably, at least one, and more preferably both, of R⁹ and R¹⁰ is H.

In some preferred embodiments, R¹¹ is phenyl-R¹⁴, wherein R¹⁴ is selected from C₁₋₃ alkyl, O-C₁₋₃ alkyl, CF₃, OCF₃ and halogen (preferably, F).

In some particularly preferred embodiments, R¹¹ is phenyl-R¹⁴, wherein R¹⁴ is selected from CH₃, OCH₃, CF₃, OCF₃, F and Cl. In such embodiments, the phenyl of R¹¹ is preferably substituted at just a single position, preferably the carbon atom at position 4.

In some preferred embodiments, the compounds of Formula I exhibit anti-proliferative activity in human cell lines, as measured by a cytotoxicity assay. Preferably, the compound exhibits an IC₅₀ value of less than 10 µM, even more preferably less than 5 µM as measured by a standard cell viability assay.

In some preferred embodiments, the compounds of Formula I inhibit one or more protein kinases, as measured by any standard assay well known to those skilled in the art. Preferably, the compound exhibits an IC₅₀ value of less than 1 µM or less than 0.5 µM as measured by the kinase assay described in Example 2 hereinafter, more preferably still less than 0.1 µM.

Particular examples of compounds according to the first aspect are shown in Table 1 below.

TABLE 1

Chemical structure of selected compounds of the present disclosure

| No. | Structure | Name | Mass |
|---|---|---|---|
| 1 | 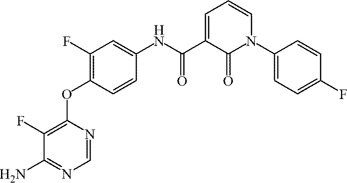 [SMILES: Nc1ncnc(Oc2ccc(NC(=O)c3cccn(-c4ccc(F)cc4)c3=O)cc2F)c1F] | N-(4-((6-amino-5-fluoropyrimidin-4-yl)oxy)-3-fluorophenyl)-1-(4-fluorophenyl)-2-oxo-1,2-dihydropyridine-3-carboxamide | 453.1 |
| 2 | 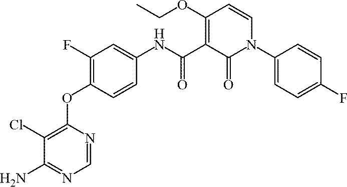 [SMILES: CCOc1ccn(-c2ccc(F)cc2)c(=O)c1C(=O)Nc1ccc(Oc2ncnc(N)c2Cl)c(F)c1] | N-(4-((6-amino-5-chloropyrimidin-4-yl)oxy)-3-fluorophenyl)-4-ethoxy- 1-(4-fluorophenyl)-2-oxo-1,2-dihydropyridine-3-carboxamide | 513.1 |
| 3 | 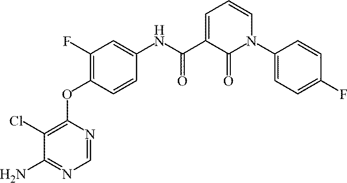 [SMILES: Nc1ncnc(Oc2ccc(NC(=O)c3cccn(-c4ccc(F)cc4)c3=O)cc2F)c1Cl] | N-(4-((6-amino-5-chloropyrimidin-4-yl)oxy)-3-fluorophenyl)-1-(4-fluorophenyl)-2-oxo-1,2-dihydropyridine-3-carboxamide | 469.1 |
| 4 | 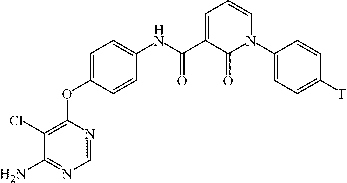 [SMILES: Nc1ncnc(Oc2ccc(NC(=O)c3cccn(-c4ccc(F)cc4)c3=O)cc2)c1Cl] | N-(4-((6-amino-5-chloropyrimidin-4-yl)oxy)phenyl)-1-(4-fluorophenyl)-2-oxo-1,2-dihydropyridine-3-carboxamide | 451.1 |
| 5 | 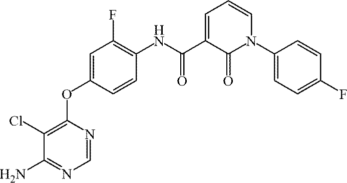 [SMILES: Nc1ncnc(Oc2ccc(NC(=O)c3cccn(-c4ccc(F)cc4)c3=O)c(F)c2)c1Cl] | N-(4-((6-amino-5-chloropyrimidin-4-yl)oxy)-2-fluorophenyl)-1-(4-fluorophenyl)-2-oxo-1,2-dihydropyridine-3-carboxamide | 469.1 |
| 6 | 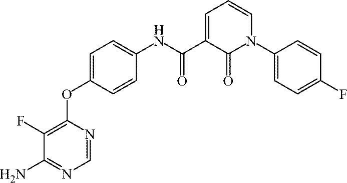 [SMILES: Nc1ncnc(Oc2ccc(NC(=O)c3cccn(-c4ccc(F)cc4)c3=O)cc2)c1F] | N-(4-((6-amino-5-fluoropyrimidin-4-yl)oxy)phenyl)-1-(4-fluorophenyl)-2-oxo-1,2-dihydropyridine-3-carboxamide | 435.1 |
| 7 | 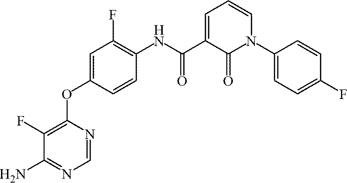 [SMILES: Nc1ncnc(Oc2ccc(NC(=O)c3cccn(-c4ccc(F)cc4)c3=O)c(F)c2)c1F] | N-(4-((6-amino-5-fluoropyrimidin-4-yl)oxy)-2-fluorophenyl)-1-(4-fluorophenyl)-2-oxo-1,2-dihydropyridine-3-carboxamide | 453.1 |
| 8 | 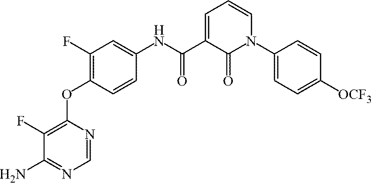 [SMILES: Nc1ncnc(Oc2ccc(NC(=O)c3cccn(-c4ccc(OC(F)(F)F)cc4)c3=O)cc2F)c1F] | N-(4-((6-amino-5-fluoropyrimidin-4-yl)oxy)-3-fluorophenyl)-2-oxo-1-(4-(trifluoromethoxy)phenyl)-1,2-dihydropyridine-3-carboxamide | 519.1 |
| 9 | 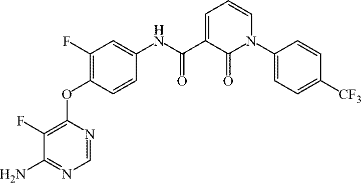 [SMILES: Nc1ncnc(Oc2ccc(NC(=O)c3cccn(-c4ccc(C(F)(F)F)cc4)c3=O)cc2F)c1F] | N-(4-((6-amino-5-fluoropyrimidin-4-yl)oxy)-3-fluorophenyl)-2-oxo-1-(4-(trifluoromethyl)phenyl)-1,2-dihydropyridine-3-carboxamide | 503.1 |
| 10 | 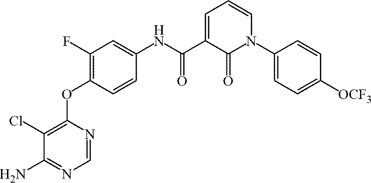 [SMILES: Nc1ncnc(Oc2ccc(NC(=O)c3cccn(-c4ccc(OC(F)(F)F)cc4)c3=O)cc2F)c1Cl] | N-(4-((6-amino-5-chloropyrimidin-4-yl)oxy)-3-fluorophenyl)-2-oxo-1-(4-(trifluoromethoxy)phenyl)-1,2-dihydropyridine-3-carboxamide | 535.1 |
| 11 | 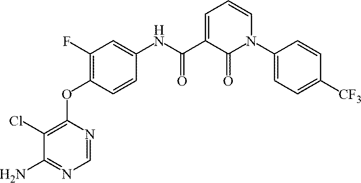 [SMILES: Nc1ncnc(Oc2ccc(NC(=O)c3cccn(-c4ccc(C(F)(F)F)cc4)c3=O)cc2F)c1Cl] | N-(4-((6-amino-5-chloropyrimidin-4-yl)oxy)-3-fluorophenyl)-2-oxo-1-(4-(trifluoromethyl)phenyl)-1,2-dihydropyridine-3-carboxamide | 519.1 |
| 12 | 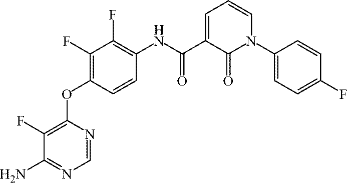 [SMILES: Nc1ncnc(Oc2ccc(NC(=O)c3cccn(-c4ccc(F)cc4)c3=O)c(F)c2F)c1F] | N-(4-((6-amino-5-fluoropyrimidin-4-yl)oxy)-2,3-difluorophenyl)-1-(4-fluorophenyl)-2-oxo-1,2-dihydropyridine-3-carboxamide | 471.1 |
| 13 | 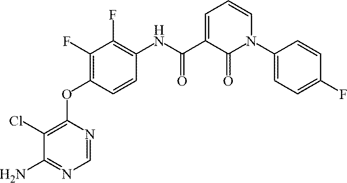 [SMILES: Nc1ncnc(Oc2ccc(NC(=O)c3cccn(-c4ccc(F)cc4)c3=O)c(F)c2F)c1Cl] | N-(4-((6-amino-5-chloropyrimidin-4-yl)oxy)-2,3-difluorophenyl)-1-(4-fluorophenyl)-2-oxo-1,2-dihydropyridine-3-carboxamide | 487.1 |
| 14 | 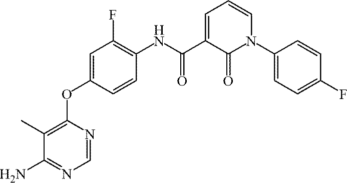 [SMILES: Cc1c(N)ncnc1Oc1ccc(NC(=O)c2cccn(-c3ccc(F)cc3)c2=O)c(F)c1] | N-(4-((6-amino-5-methylpyrimidin-4-yl)oxy)-2-fluorophenyl)-1-(4-fluorophenyl)-2-oxo-1,2-dihydropyridine-3-carboxamide | 449.1 |
| 15 | 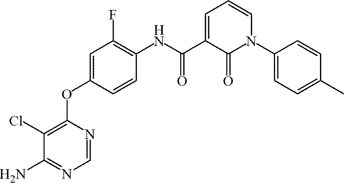 [SMILES: Cc1ccc(-n2cccc(C(=O)Nc3ccc(Oc4ncnc(N)c4Cl)cc3F)c2=O)cc1] | N-(4-((6-amino-5-chloropyrimidin-4-yl)oxy)-2-fluorophenyl)-2-oxo-1-(p-tolyl)-1,2-dihydropyridine-3-carboxamide | 465.9 |
| 16 | 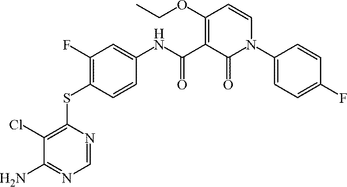 [SMILES: CCOc1ccn(-c2ccc(F)cc2)c(=O)c1C(=O)Nc1ccc(Sc2ncnc(N)c2Cl)c(F)c1] | N-(4-((6-amino-5-chloropyrimidin-4-yl)thio)-3-fluorophenyl)-4-ethoxy-1-(4-fluorophenyl)-2-oxo-1,2-dihydropyridine-3-carboxamide | 529.9 |
| 17 | 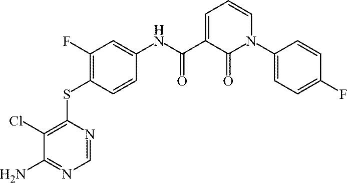 [SMILES: Nc1ncnc(Sc2ccc(NC(=O)c3cccn(-c4ccc(F)cc4)c3=O)cc2F)c1Cl] | N-(4-((6-amino-5-chloropyrimidin-4-yl)thio)-3-fluorophenyl)-1-(4-fluorophenyl)-2-oxo-1,2-dihydropyridine-3-carboxamide | 485.1 |
| 18 | 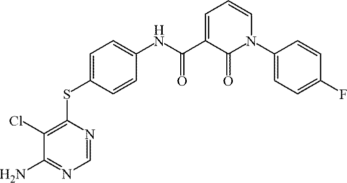 [SMILES: Nc1ncnc(Sc2ccc(NC(=O)c3cccn(-c4ccc(F)cc4)c3=O)cc2)c1Cl] | N-(4-((6-amino-5-chloropyrimidin-4-yl)thio)phenyl)-1-(4-fluorophenyl)-2-oxo-1,2-dihydropyridine-3-carboxamide | 467.1 |
| 19 | 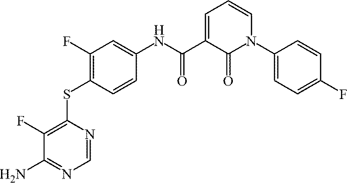 [SMILES: Nc1ncnc(Sc2ccc(NC(=O)c3cccn(-c4ccc(F)cc4)c3=O)cc2F)c1F] | N-(4-((6-amino-5-fluoropyrimidin-4-yl)thio)-3-fluorophenyl)-1-(4-fluorophenyl)-2-oxo-1,2-dihydropyridine-3-carboxamide | 469.1 |
| 20 | 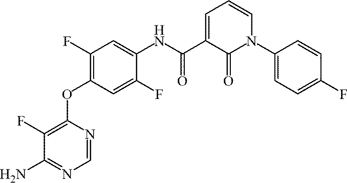 [SMILES: Nc1ncnc(Oc2cc(F)c(NC(=O)c3cccn(-c4ccc(F)cc4)c3=O)cc2F)c1F] | N-(4-((6-amino-5-fluoropyrimidin-4-yl)oxy)-2,5-difluorophenyl)-1-(4-fluorophenyl)-2-oxo-1,2-dihydropyridine-3-carboxamide | 471.4 |
| 21 | 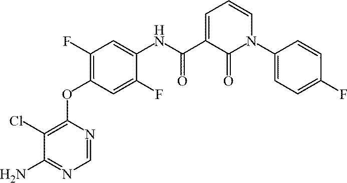 [SMILES: Nc1ncnc(Oc2cc(F)c(NC(=O)c3cccn(-c4ccc(F)cc4)c3=O)cc2F)c1Cl] | N-(4-((6-amino-5-chloropyrimidin-4-yl)oxy)-2,5-difluorophenyl)-1-(4-fluorophenyl)-2-oxo-1,2-dihydropyridine-3-carboxamide | 487.8 |
| 22 | 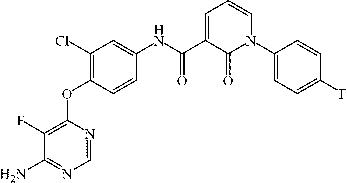 [SMILES: Nc1ncnc(Oc2ccc(NC(=O)c3cccn(-c4ccc(F)cc4)c3=O)cc2Cl)c1F] | N-(4-((6-amino-5-fluoropyrimidin-4-yl)oxy)-3-chlorophenyl)-1-(4-fluorophenyl)-2-oxo-1,2-dihydropyridine-3-carboxamide | 469.8 |
| 23 | 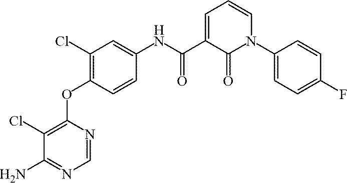 [SMILES: Nc1ncnc(Oc2ccc(NC(=O)c3cccn(-c4ccc(F)cc4)c3=O)cc2Cl)c1Cl] | N-(4-((6-amino-5-chloropyrimidin-4-yl)oxy)-3-chlorophenyl)-1-(4-fluorophenyl)-2-oxo-1,2-dihydropyridine-3-carboxamide | 486.3 |
| 24 | 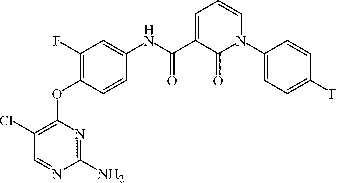 [SMILES: Nc1ncc(Cl)c(Oc2ccc(NC(=O)c3cccn(-c4ccc(F)cc4)c3=O)cc2F)n1] | N-(4-((2-amino-5-chloropyrimidin-4-yl)oxy)-3-fluorophenyl)-1-(4-fluorophenyl)-2-oxo-1,2-dihydropyridine-3-carboxamide | 469.8 |
| 25 | 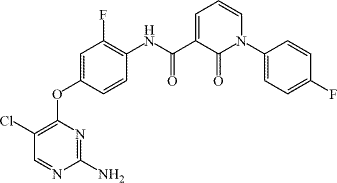 [SMILES: Nc1ncc(Cl)c(Oc2ccc(NC(=O)c3cccn(-c4ccc(F)cc4)c3=O)c(F)c2)n1] | N-(4-((2-amino-5-chloropyrimidin-4-yl)oxy)-2-fluorophenyl)-1-(4-fluorophenyl)-2-oxo-1,2-dihydropyridine-3-carboxamide | 469.8 |
| 26 | 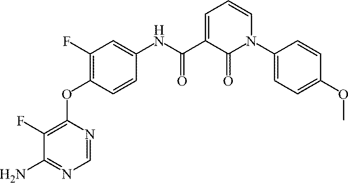 [SMILES: COc1ccc(-n2cccc(C(=O)Nc3ccc(Oc4ncnc(N)c4F)c(F)c3)c2=O)cc1] | N-(4-((6-amino-5-fluoropyrimidin-4-yl)oxy)-3-fluorophenyl)-1-(4-methoxyphenyl)-2-oxo-1,2-dihydropyridine-3-carboxamide | 465.4 |
| 27 | 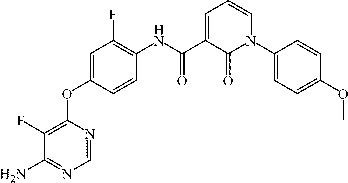 [SMILES: COc1ccc(-n2cccc(C(=O)Nc3ccc(Oc4ncnc(N)c4F)cc3F)c2=O)cc1] | N-(4-((6-amino-5-fluoropyrimidin-4-yl)oxy)-2-fluorophenyl)-1-(4-methoxyphenyl)-2-oxo-1,2-dihydropyridine-3-carboxamide | 465.4 |
| 28 | 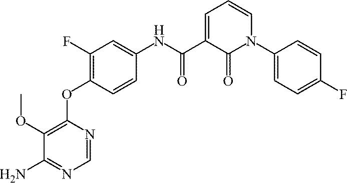 [SMILES: COc1c(N)ncnc1Oc1ccc(NC(=O)c2cccn(-c3ccc(F)cc3)c2=O)cc1F] | N-(4-((6-amino-5-methoxypyrimidin-4-yl)oxy)-3-fluorophenyl)-1-(4-fluorophenyl)-2-oxo-1,2-dihydropyridine-3-carboxamide | 465.4 |
| 29 | 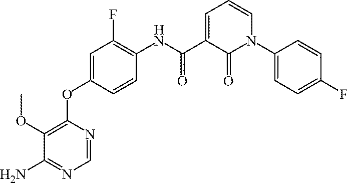 [SMILES: COc1c(N)ncnc1Oc1ccc(NC(=O)c2cccn(-c3ccc(F)cc3)c2=O)c(F)c1] | N-(4-((6-amino-5-methoxypyrimidin-4-yl)oxy)-2-fluorophenyl)-1-(4-fluorophenyl)-2-oxo-1,2-dihydropyridine-3-carboxamide | 465.4 |
| 30 | 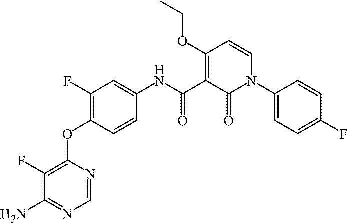 [SMILES: CCOc1ccn(-c2ccc(F)cc2)c(=O)c1C(=O)Nc1ccc(Oc2ncnc(N)c2F)c(F)c1] | N-(4-((6-amino-5-fluoropyrimidin-4-yl)oxy)-3-fluorophenyl)-4-ethoxy-1-(4-fluorophenyl)-2-oxo-1,2-dihydropyridine-3-carboxamide | 497.4 |
| 31 | 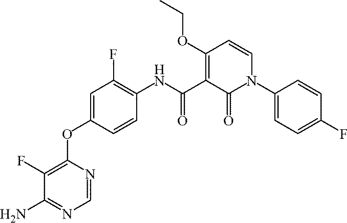 [SMILES: CCOc1ccn(-c2ccc(F)cc2)c(=O)c1C(=O)Nc1ccc(Oc2ncnc(N)c2F)cc1F] | N-(4-((6-amino-5-fluoropyrimidin-4-yl)oxy)-2-fluorophenyl)-4-ethoxy-1-(4-fluorophenyl)-2-oxo-1,2-dihydropyridine-3-carboxamide | 497.4 |
| 32 | 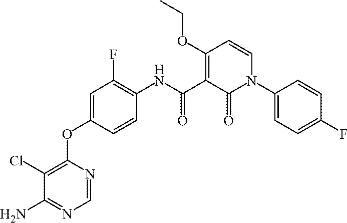 [SMILES: CCOc1ccn(-c2ccc(F)cc2)c(=O)c1C(=O)Nc1ccc(Oc2ncnc(N)c2Cl)cc1F] | N-(4-((6-amino-5-chloropyrimidin-4-yl)oxy)-2-fluorophenyl)-4-ethoxy-1-(4-fluorophenyl)-2-oxo-1,2-dihydropyridine-3-carboxamide | 513.9 |
| 33 | 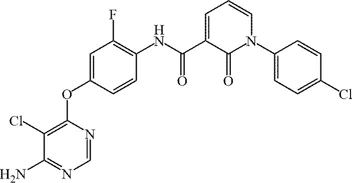 [SMILES: Nc1ncnc(Oc2ccc(NC(=O)c3cccn(-c4ccc(Cl)cc4)c3=O)c(F)c2)c1Cl] | N-(4-((6-amino-5-chloropyrimidin-4-yl)oxy)-2-fluorophenyl)-1-(4-chlorophenyl)-2-oxo-1,2-dihydropyridine-3-carboxamide | 486.3 |
| 34 | 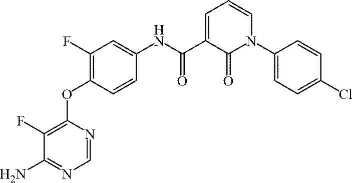 [SMILES: Nc1ncnc(Oc2ccc(NC(=O)c3cccn(-c4ccc(Cl)cc4)c3=O)cc2F)c1F] | N-(4-((6-amino-5-fluoropyrimidin-4-yl)oxy)-3-fluorophenyl)-1-(4-chlorophenyl)-2-oxo-1,2-dihydropyridine-3-carboxamide | 469.8 |
| 35 | 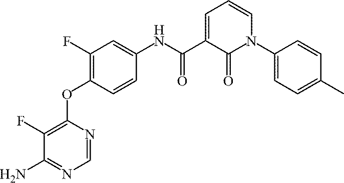 [SMILES: Cc1ccc(-n2cccc(C(=O)Nc3ccc(Oc4ncnc(N)c4F)c(F)c3)c2=O)cc1] | N-(4-((6-amino-5-fluoropyrimidin-4-yl)oxy)-3-fluorophenyl)-2-oxo-1-(p-tolyl)-1,2-dihydropyridine-3-carboxamide | 449.4 |
| 36 | 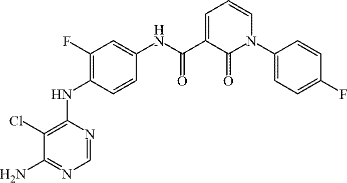 [SMILES: Nc1ncnc(Nc2ccc(NC(=O)c3cccn(-c4ccc(F)cc4)c3=O)cc2F)c1Cl] | N-(4-((6-amino-5-chloropyrimidin-4-yl)amino)-3-fluorophenyl)-1-(4-fluorophenyl)-2-oxo-1,2-dihydropyridine-3-carboxamide | 468.8 |
| 37 | 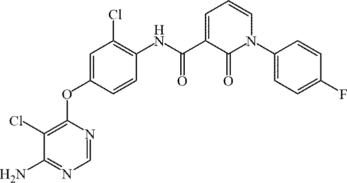 [SMILES: Nc1ncnc(Oc2ccc(NC(=O)c3cccn(-c4ccc(F)cc4)c3=O)c(Cl)c2)c1Cl] | N-(4-((6-amino-5-chloropyrimidin-4-yl)oxy)-2-chlorophenyl)-1-(4-fluorophenyl)-2-oxo-1,2-dihydropyridine-3-carboxamide | 486.3 |
| 38 | 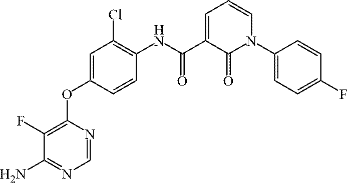 [SMILES: Nc1ncnc(Oc2ccc(NC(=O)c3cccn(-c4ccc(F)cc4)c3=O)c(Cl)c2)c1F] | N-(4-((6-amino-5-fluoropyrimidin-4-yl)oxy)-2-chlorophenyl)-1-(4-fluorophenyl)-2-oxo-1,2-dihydropyridine-3-carboxamide | 469.8 |
| 39 | 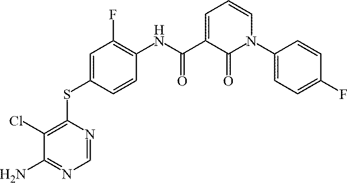 [SMILES: Nc1ncnc(Sc2ccc(NC(=O)c3cccn(-c4ccc(F)cc4)c3=O)c(F)c2)c1Cl] | N-(4-((6-amino-5-chloropyrimidin-4-yl)thio)-2-fluorophenyl)-1-(4-fluorophenyl)-2-oxo-1,2-dihydropyridine-3-carboxamide | 485.9 |

The compounds (and pharmaceutically acceptable salts, solvates and prodrugs thereof) may be administered in combination with one or more additional agent(s) for the treatment of cancer or another proliferative disease or condition. For example, the compounds may be used in combination with other anti-cancer agents in order to inhibit more than one cancer signalling pathway simultaneously so as to make cancer cells more susceptible to anti-cancer therapies (eg treatments with other anti-cancer agents, chemotherapy, radiotherapy or a combination thereof). As such, the compounds of Formula I may be used in combination with one or more of the following categories of anti-cancer agents:
other anti-proliferative/antineoplastic drugs and combinations thereof, as used in medical oncology, such as alkylating agents (eg cis-platin, oxaliplatin, carboplatin, cyclophosphamide, nitrogen mustard, melphalan, chlorambucil, busulphan, temozolamide and nitrosoureas); antimetabolites (eg gemcitabine and antifolates such as fluoropyrimidines like 5-fluorouracil and tegafur, raltitrexed, methotrexate, cytosine arabinoside, fludarabine and hydroxyurea); antitumour antibiotics (eg anthracyclines such as adriamycin, bleomycin, doxorubicin, daunomycin, epirubicin, idarubicin, mitomycin-C, dactinomycin and mithramycin); antimitotic agents (eg vinca alkaloids such as vincristine, vinblastine, vindesine and vinorelbine and taxoids including taxol and taxotere and polokinase inhibitors); and topoisomerase inhibitors (eg epipodophyllotoxins such as etoposide and teniposide, amsacrine, topotecan and camptothecin);
cytostatic agents such as antioestrogens (eg tamoxifen, fulvestrant, toremifene, raloxifene, droloxifene and iodoxyfene), antiandrogens (eg bicalutamide, flutamide, nilutamide and cyproterone acetate), LHRH antagonists or LHRH agonists (eg goserelin, leuprorelin and buserelin), progestogens (eg megestrol acetate), aromatase inhibitors (eg as anastrozole, letrozole, vorazole and exemestane) and inhibitors of 5α-reductase such as finasteride;
anti-invasion agents (eg c-Src kinase family inhibitors such as 4-(6-chloro-2,3-methylenedioxyanilino)-7-[2-(4-methylpiperazin-1-yl)ethoxy]-5-tetrahydropyran-4-yloxyquinazoline (AZD0530; International Patent Publication No WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib) and bosutinib (SKI-606)), and metalloproteinase inhibitors including marimastat, inhibitors of urokinase plasminogen activator receptor function or antibodies to heparanase;
inhibitors of growth factor function (eg growth factor antibodies and growth factor receptor antibodies such as the anti-erbB2 antibody trastuzumab (Herceptin™), the anti-EGFR antibody panitumumab, the anti-erbB1 antibody cetuximab (Erbitux, C225) and any growth factor or growth factor receptor antibodies disclosed by Stern et al. Critical reviews in oncology/haematology, 2005, Vol. 54, pp11-29). Such inhibitors also include tyrosine kinase inhibitors such as inhibitors of the epidermal growth factor family (eg EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (eg Ras/Raf signalling inhibitors such as farnesyl transferase inhibitors including sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (eg AZD1152, PH739358, VX-680, MLN8054, R763, MP235, MP529, VX-528 and AX39459) and cyclin dependent kinase inhibitors such as CDK2 and/or CDK9 inhibitors;
antiangiogenic agents such as those which inhibit the effects of vascular endothelial growth factor (eg the anti-vascular endothelial cell growth factor antibody bevacizumab (Avastin™) and VEGF receptor tyrosine kinase inhibitors such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within International Patent Publication No WO 00/47212), compounds such as those disclosed in International Patent Publication Nos WO97/22596, WO 97/30035, WO 97/32856 and WO 98/13354, and compounds that work by other mechanisms (eg linomide, inhibitors of integrin αvβ3 function and angiostatin);
vascular damaging agents such as Combretastatin A4 and compounds disclosed in International Patent Publication Nos WO 99/02166, WO 00/40529, WO 00/41669, WO 01/92224, WO 02/04434 and WO 02/08213;
an endothelin receptor antagonist such as zibotentan (ZD4054) or atrasentan;
antisense therapies such as those which are directed to the targets listed above, such as ISIS 2503, an anti-ras antisense;
gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex vivo and in vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Where used in combination with other anti-cancer agents, a compound of Formula I and the other anti-cancer agent can be administered in the same pharmaceutical composition or in separate pharmaceutical compositions. If administered in separate pharmaceutical compositions, the compound and the other anti-cancer agent may be administered simultaneously or sequentially in any order (eg within seconds or minutes or even hours (eg 2 to 48 hours)).

The compound of the Formula I is typically applied to the treatment of cancer or another proliferative cell disease or condition in a human subject. However, the subject may also be selected from, for example, livestock animals (eg cows, horses, pigs, sheep and goats), companion animals (eg dogs and cats) and exotic animals (eg non-human primates, tigers, elephants etc).

Cancers and other proliferative cell diseases and conditions that may be treated in accordance with the present disclosure include biliary tract cancer, brain cancer and other cancers of the central nervous system (CNS) (including glioblastomas and medulloblastomas), neuroblastomas, breast cancer, cervical cancer, ovarian cancer (including those arising from epithelial cells, stromal cells, germ cells, and mesenchymal cells), choriocarcinoma, colorectal cancer, endometrial cancer, liver cancer, lung cancer, oesophageal cancer, gastric cancer, haematological neoplasms (including acute lymphocytic leukaemia (ALL)), chronic lymphocytic leukaemia (CLL) and chronic myelogenous leukaemia (CML), and acute myeloid leukaemia (AML), multiple myeloma, AIDS-associated leukaemia’s and adult T-cell leukaemia lymphoma, lymphomas (including Non-Hodgkin’s lymphoma, Hodgkin’s disease and lymphocytic lymphomas)), intraepithelial neoplasms (including Bowen’s disease and Paget’s disease), oral cancer (including squamous cell carcinoma), pancreatic cancer, prostate cancer, sarcomas (including leiomyosarcoma, rhabdomyosarcoma, liposarcoma, fibrosarcoma, and osteosarcoma), skin cancer (including melanoma, Kaposi’s sarcoma, basocellular cancer, and squamous cell cancer), testicular cancer (including germinal tumours such as seminoma, non-seminoma teratomas, and choriocarcinomas), stromal tumours, germ cell tumours, thyroid cancer (including thyroid adenocarcinoma and medullar carcinoma), and renal cancer (including adenocarcinoma and Wilms’ tumour).

In some embodiments, the compounds of Formula I are used to treat cancers or other conditions dependent upon TAM and/or MET activation, wherein the TAM and/or MET activation may be regulated by gene amplification or activated TAM and/or MET mutant forms.

The compounds of Formula I may be formulated into a pharmaceutical composition with a pharmaceutically acceptable carrier, diluent and/or excipient. Examples of suitable carriers and diluents are well known to those skilled in the art, and are described in, for example, Remington’s Pharmaceutical Sciences, Mack Publishing Co., Easton, PA 1995. Examples of suitable excipients for the various different forms of pharmaceutical compositions described herein may be found in the Handbook of Pharmaceutical Excipients, 2^{nd} Edition, (1994), Edited by A Wade and PJ Weller. Examples of suitable carriers include lactose, starch, glucose, methyl cellulose, magnesium stearate, mannitol, sorbitol and the like. Examples of suitable diluents include ethanol, glycerol and water. The choice of carrier, diluent and/or excipient may be made with regard to the intended route of administration and standard pharmaceutical practice.

A pharmaceutical composition comprising a compound of Formula I may further comprise any suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilising agents, dyes and even flavouring agents may be provided in the pharmaceutical composition. Examples of preservatives include sodium benzoate, sorbic acid and esters of p-hydroxybenzoic acid. Anti-oxidants and suspending agents may be also used.

A pharmaceutical composition comprising a compound of Formula I may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration. For oral administration, particular use may be made of compressed tablets, pills, tablets, gellules, drops, and capsules. For other forms of administration, a pharmaceutical composition may comprise solutions or emulsions which may be injected intravenously, intraarterially, intrathecally, subcutaneously, intradermally, intraperitoneally or intramuscularly, and which are prepared from sterile or sterilisable solutions. A pharmaceutical composition comprising a compound of Formula I may also be in form of suppositories, pessaries, suspensions, emulsions, lotions, ointments, creams, gels, sprays, solutions or dusting powders. A pharmaceutical composition may be formulated in unit dosage form (ie in the form of discrete portions containing a unit dose, or a multiple or sub-unit of a unit dose).

The compounds of Formula I may be provided as a pharmaceutically acceptable salt including, for example, suitable acid addition or base salts thereof. A review of suitable pharmaceutical salts may be found in Berge et al., J Pharm Sci 66:1-19 (1977). Salts are formed, for example with strong inorganic acids such as mineral acids (eg sulfuric acid, phosphoric acid or hydrohalic acids), with strong organic carboxylic acids, such as alkanecarboxylic acids of 1 to 4 carbon atoms which are unsubstituted or substituted (eg by halogen), such as acetic acid, with saturated or unsaturated dicarboxylic acids (eg oxalic, malonic, succinic, maleic, fumaric, phthalic or tetraphthalic acid), with hydroxycarboxylic acids (eg ascorbic, glycolic, lactic, malic, tartaric or citric acid), with amino acids (eg aspartic or glutamic acid), with benzoic acid, or with organic sulfonic acids (eg (C₁-C₄)-alkyl- or aryl-sulfonic acids which are unsubstituted or substituted by, for example, halogen) such as methane- or p-toluene sulfonic acid).

The compounds of Formula I may be provided in their various crystalline forms, polymorphic forms and (an)hydrous forms. In this regard, it is well known to those skilled in the art that chemical compounds may be isolated in any of such forms by slightly varying the method of purification and or isolation from the solvents used in the synthetic preparation of such compounds.

The present disclosure further provides a method of synthesising a compound according to Formula I, or a pharmaceutically acceptable salt, solvate or prodrug thereof.

With regard to the description of the synthetic methods described below and in the referenced synthetic methods that are used to prepare starting materials, it will be understood by those skilled in the art that all proposed reaction conditions, including choice of solvent, reaction atmosphere, reaction temperature, duration of the experiment and workup procedures, can be readily selected. Moreover, it will be understood by those skilled in the art that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the examples hereinafter. Alternatively, necessary starting materials may be obtainable by analogous procedures to those illustrated which are within the ordinary skill of those skilled in the art. Further, it will be appreciated that during the synthesis of the compounds, in the processes described below, or during the synthesis of certain starting materials, it may be desirable to protect certain substituent groups to prevent their undesired reaction. Those skilled in the art will readily recognise when such protection is required, and how such protecting groups may be put in place, and later removed. Examples of protecting groups are described in, for example, Protective Groups in Organic Synthesis by Theodora Green (publisher: John Wiley & Sons). Protecting groups may be removed by any convenient method well known to those skilled in the art as appropriate for the removal of the protecting group in question, such methods being chosen so as to effect removal of the protecting group with the minimum disturbance of groups elsewhere in the molecule. Thus, if reactants include, for example, groups such as amino, carboxyl or hydroxyl, it may be desirable to protect the group in some of the reactions mentioned herein.

Synthetic methodologies for the preparation of compounds of Formula I will be readily apparent to those skilled in the art.

However, in a further aspect of the present disclosure, a method of synthesising a compound of Formula I (or a pharmaceutically acceptable salt, solvate or prodrug thereof) is provided wherein the method comprises:
a) reacting a compound of formula A:

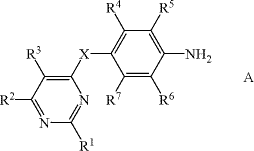

wherein
   X is O or S;
   R¹, R², R³, R⁴, R⁵, R⁶ and R⁷ are as defined above in respect of Formula I, with a suitable 2-oxo-1,2-dihydropyridine-3-carboxylic acid derivative reacting a compound of formula B: 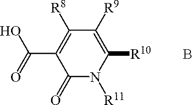
   wherein R⁸, R⁹, R¹⁰ and R¹¹ are as defined above in respect of Formula I, and if necessary
b) removing any protecting groups present, and/or forming a pharmaceutically acceptable salt, solvate or prodrug thereof.

In still a further aspect of the present disclosure, a method of synthesising a compound of Formula I (or a pharmaceutically acceptable salt, solvate or prodrug thereof) is provided wherein the method comprises:
a) reacting a compound of formula B:

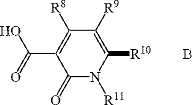

wherein R⁸, R⁹, R¹⁰ and R¹¹ are as defined above in respect of Formula I, with a compound having the following formula C: 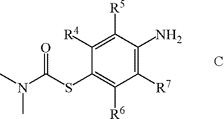
wherein R⁴, R⁵, R⁶ and R⁷ are as defined above in respect of Formula I to provide a compound of formula D 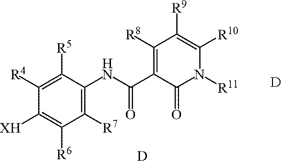
b) reacting the compound of formula D with a halogenated pyrimidine and, if necessary
c) removing any protecting groups present, and/or forming a pharmaceutically acceptable salt, solvate or prodrug thereof.

The coupling reaction between the compounds of formula A and formula B may take place in the presence of a suitable solvent or solvent mixture. Those skilled in the art will be able to readily select a suitable solvent or solvent mixture for use in this reaction. Examples of suitable solvents include acetonitrile, halogenated solvents, etc.

In addition, those skilled in the art will be able to select appropriate reaction conditions to use in the coupling reaction of the compound of formula A and formula B. However, typically, the reaction will be carried out in anhydrous conditions and in the presence of an inert atmosphere, such as argon or nitrogen. The reaction may also be carried out at room temperature or an elevated temperature for a suitable time period of, for example, 30 minutes to 48 hours.

The resultant compound can be isolated and purified using techniques well known to those skilled in the art.

An example of a particularly suitable method for synthesising a compound of the present disclosure is shown as Scheme 1 below.

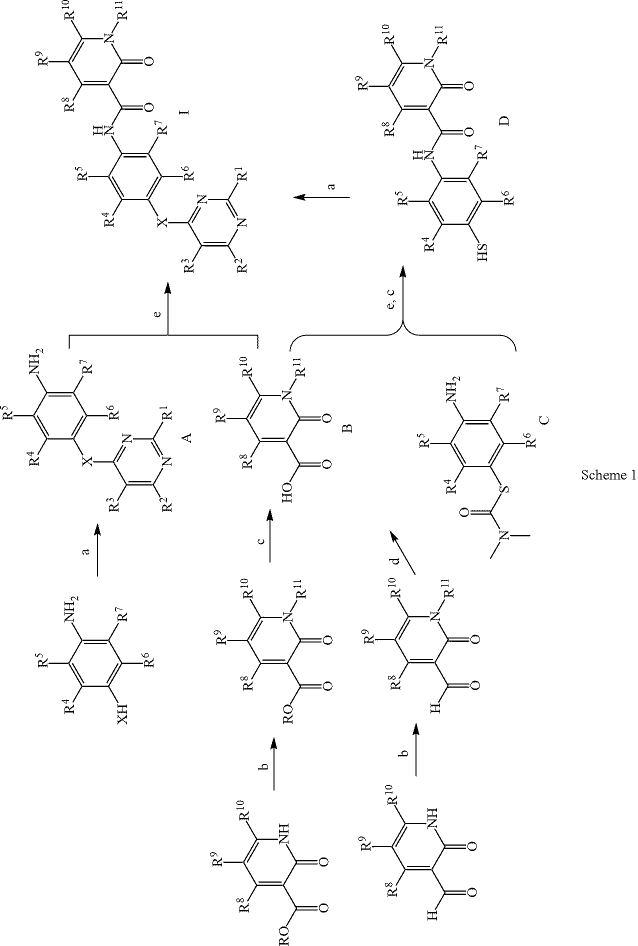

wherein the general reaction conditions are: (a) appropriate halogenated pyrimidine, K₂CO₃ or Cs₂CO₃, DMF, rt - 80° C., 12-24 h; (b) appropriate boronic acid, Cu(CH₃CO₂)₂, pyridine, rt; (c) LiOH, THF/MeOH/H₂O (2:2:1), rt - 80° C., 12-24 h; (d) NaClO₂, NaH₂PO₄, 2-methyl-2-butene, THF/t-butanol/H₂O (1:1:1), 0° C. - rt, 0.5 - 2 h; and (e) HATU, DIPEA, rt, 2-4 h; or SOCl₂, DIPEA, 0° C. - rt, 0.5-2 h.

The disclosure is hereinafter described with reference to the following, non-limiting examples and accompanying figures.

### EXAMPLES

### Example 1 Synthesis

### General

¹H and ¹³C NMR spectra were recorded at 298 K (unless otherwise specified) on a Bruker AVANCE III HD 500 spectrometer (¹H at 500.20 MHz and ¹³C at 125.79 MHz), and were analysed using Bruker Topspin 3.2 software. ¹H NMR signals are reported with chemical shift values δ (ppm), multiplicity (s = singlet, d = doublet, t = triplet, q = quartet, dd = doublet of doublets, dt = doublet of triplets, td = triplet of doublets, ddd = doublet of doublet of doublets, m = multiplet and br = broad), relative integral, coupling constants J (Hz) and assignments. High resolution mass spectra were recorded on an AB SCIEX TripleTOF 5600 mass spectrometer (Concord, ON, Canada), and ionisation of all samples was carried out using ESI.

General Synthetic Procedures: To a solution of carboxylic acid B (1.15 equiv) and HATU (1.2 equiv) in DCM under N₂ was added DIPEA (1.2 equiv) and the reaction mixture was stirred for 15 min under room temperature. A solution of an appropriate arylamine A (1 equiv) in DCM was then added and the reaction mixture was stirred for 4 h under room temperature. The reaction mixture was then concentrated under reduced pressure. The residue was dissolved in DCM and washed with saturated NH₄Cl solution. The organic phase was dried over MgSO₄ and concentrated under reduced pressure. The residue was purified by flash chromatography (silica gel). The resulting product was dissolved in a solution of DCM/TFA (1:1) and the mixture was stirred for 2 h under room temperature. The reaction mixture was then concentrated under reduced pressure. The residue was dissolved in DCM and washed with 1 M NaOH solution. The organic phase was dried over MgSO₄ and concentrated under reduced pressure to give the desired compounds.

### Examples

### N-((6-amino-5-fluoropyrimidin-4-yl)oxy)-3-fluorophenyl)-1-fluorophenyl)-2-oxo-1,2-dihydropyridine-3-carboxamide (1)

A mixture of methyl 2-oxo-1,2-dihydropyridine-3-carboxylate (500 mg, 3.27 mmol), 4-fluorophenyl boronic acid (1.37 g, 9.80 mmol), copper (II) acetate (1.19 g, 6.55 mmol) in DCM (25 mL) and pyridine (1.1 mL, 13.7 mmol) was stirred in the presence of air at room temperature for 18 h. The reaction mixture was filtered through a pad of celite and the filtrate concentrated under reduced pressure. The residue was dissolved in EtOAc (100 mL) and washed with 1 M HCl aqueous solution (50 mL). The organic phase was washed with brine (50 mL), dried over MgSO₄ and concentrated. The residue was purified by flash chromatography (silica, PE ramping to EtOAc) to offer methyl 1-(4-fluorophenyl)-2-oxo-1,2-dihydropyridine-3-carboxylate as a white solid (394 mg, 49%). ¹H NMR (MeOD) δ 3.78 (s, 3H), 6.45 (t, 1H, J = 7.0 Hz), 7.20 (t, 2H, J = 8.0 Hz), 7.38 (m, 2H), 7.81 (d, 1H, J = 6.5 Hz), 8.23 (d, 1H, J = 7.0 Hz). HRMS m/z 248.0841 [M+H]⁺.

Then, to a solution of methyl 1-(4-fluorophenyl)-2-oxo-1,2-dihydropyridine-3-carboxylate (500 mg, 2.02 mmol) and lithium hydroxide (100 mg, 4.18 mmol) in THF/MeOH (1:1, 4 mL) was added with H₂O (1 mL). After stirred for 3 h, the reaction mixture was quenched with 1 M HCl in water (2 mL) and concentrated under reduced pressure. The residue was dissolved in EtOAc (50 mL) and washed with 1 M HCl aqueous solution. The aqueous phase was extracted with EtOAc (2 × 50 mL). The organic extracts were combined, dried under MgSO₄ and concentrated under reduced pressure to give 1-(4-fluorophenyl)-2-oxo-1,2-dihydropyridine-3-carboxylic acid as a white solid (463 mg, 98%). ¹H NMR (DMSO) δ 6.79 (t, 1H, J = 7.0 Hz), 7.43 (t, 2H, J = 8.5 Hz), 7.62 (dd, 2H, J = 8.5 & 8.5 Hz), 8.21 (d, 1H, J = 6.5 Hz), 8.49 (d, 1H, J = 7.0 Hz). HRMS m/z 240.0853 [M+H]⁺.

To a mixture of 6-chloro-5-fluoropyrimidin-4-amine (500 mg, 3.39 mmol), Et₃N (1 mL, 7.17 mmol) and DMAP (80 mg, 0.655 mmol) in DCM (20 mL) was added with a solution of di-tert-butyl dicarbonate (1.50 g, 6.87 mmol) in DCM (2 mL). After stirred under room temperature for 12 h, the reaction mixture was washed with 0.1 M HCl aqueous solution. The organic phase was dried over MgSO₄ and concentrated under reduced pressure. The residue was purified by flash chromatography (silica, PE ramping to PE:EtOAc = 7:3) to provide 6-chloro-5-fluoro-N,N-di-tert-butoxycarbonyl pyrimidin-4-amine as a white solid (808 mg, 69%). ¹H NMR (DMSO) δ 1.42 (s, 18H), 8.94 (s, 1H).

Then, a mixture of 6-chloro-5-fluoro-N,N-di-tert-butoxycarbonylpyrimidin-4-amine (700 mg, 2.01 mmol) and caesium carbonate (790 mg, 2.42 mmol) in DMF (5 mL) was added 4-amino-2-fluorophenol (307 mg, 2.42 mmol) and the reaction mixture was stirred for 12 h under room temperature. After concentrated under reduced pressure, the residue was added with H₂O and extracted with DCM (3 × 50 mL). The combined organic phase was dried over MgSO₄, concentrated and purified by flash chromatography (silica, PE ramping to PE:EtOAc = 2:3) to offer 6-(4-amino-2-fluorophenoxy)-5-fluoro-N,N-di-tert-butoxycarbonylpyrimidin-4-amine as a light pink powder (418 mg, 47%). ¹H NMR (CDCl₃) δ 1.48 (s, 18H), 3.80 (s, 2H), 6.50 (m, 2H), 7.03 (t, 1H, J = 8.5 Hz), 8.39 (s, 1H). HRMS m/z 439.1923 [M+H]⁺.

A mixture of 1-(4-fluorophenyl)-2-oxo-1,2-dihydropyridine-3-carboxylic acid (100 mg, 0.429 mmol), DIPEA (100 µL, 0.574 mmol) and HATU (170 mg, 0.447 mmol) in DCM (3 mL) was stirred for 15 min at room temperature. A solution of 6-(4-amino-2-fluorophenoxy)-5-fluoro-N,N-di-tert-butoxycarbonylpyrimidin-4-amine (160 mg, 0.365 mmol) in DCM (3 mL) was then added and the reaction mixture was stirred for 4 h at room temperature. The reaction mixture was added DCM (150 mL) and washed with saturated NH₄Cl solution (50 mL). The organic phase was dried over MgSO₄, concentrated and purified by flash chromatography (silica, PE ramping to EtOAc). The resulting product was treated with DCM/TFA (1:1, 6 mL) for 2 h. The reaction mixture was then concentrated under reduced pressure. The residue was dissolved in DCM (100 mL) and washed with 1 M NaOH solution. The organic phase was dried over MgSO₄ and concentrated under reduced pressure to give N-(4-((6-amino-5-fluoropyrimidin-4-yl)oxy)-3-fluorophenyl)-1-(4-fluorophenyl)-2-oxo-1,2-dihydropyridine-3-carboxamide (4) as a white solid (126 mg, 76%). ¹H NMR (CDCl₃) δ 5.03 (s, 2H), 6.61 (t, 1H, J = 7.0 Hz), 7.27 (t, 2H, J = 8.5 Hz), 7.34 (d, 1H, J = 9.0 Hz), 7.40 (m, 2H), 7.61 (dd, 1H, J = 2.0 & 6.5 Hz), 7.92 (dd, 1H, J = 2.0 & 12.5 Hz), 7.96 (s, 1H), 8.74 (dd, 1H, J = 2.0 & 7.5 Hz), 11.95 (s, 1H). HRMS m/z 454.1190 [M+H]⁺

### N-(4-((6-amino-5-chloropyrimidin-4-yl)oxy)-3-fluorophenyl)-4-ethoxy-1-(4-fluorophenyl)-2-oxo-1,2-dihydropyridine-3-carboxamide (2)

To a solution of 1-(4-fluorophenyl)-4-iodo-2-oxo-1,2-dihydropyridine-3-carboxylic acid (600 mg, 1.67 mmol) in toluene (5 mL) was treated with thionyl chloride (5 mL). After stirred for 3 h at room temperature, the reaction mixture was concentrated under reduced pressure. The residue was dissolved in DCM (6 mL) and added to a solution of 6-(4-amino-2-fluorophenoxy)-5-chloro-N,N-di-tert-butoxycarbonylpyrimidin-4-amine (476 mg, 1.05 mmol), DIPEA (0.4 mL) in DMF (0.3 mL) and THF (5 mL) in an ice bath. After 5 min, the reaction mixture was stirred for further 30 min at room temperature. The reaction mixture was quenched with saturated NaHCO₃ solution (20 mL) and the suspension was extracted with of EtOAc (2 × 100 mL). The organic phase was dried over MgSO₄, concentrated and purified by flash chromatography (silica, PE ramping to EtOAc:PE = 1:1) to offer N-(4-((5-chloro-6-(di-tert-butoxycarbonylamino)pyrimidin-4-yl)oxy)-3-fluorophenyl)-1-(4-fluorophenyl)-4-iodo-2-oxo-1,2-dihydropyridine-3-carboxamide as a light yellow powder (378 mg, 36%). ¹H NMR (CDCl₃) δ 1.45 (s, 18H), 7.12 (d, 1H, J = 7.0 Hz), 7.22 (m, 3H), 7.36 (m, 3H), 7.90 (d, 1H, J = 12.0 Hz), 8.50 (s, 1H), 11.58 (s, 1H). MS m/z 796.3 [M+H]+.

Then, to a solution of N-(4-((5-chloro-6-(di-tert-butoxycarbonylamino)pyrimidin-4-yl)oxy)-3-fluorophenyl)-1-(4-fluorophenyl)-4-iodo-2-oxo-1,2-dihydropyridine-3-carboxamide (228 mg, 0.286 mmol) in anhydrous EtOH (10 mL) was added sodium ethoxide (30 mg, 0.441 mmol) and the reaction mixture was stirred for 12 h at room temperature. The reaction mixture was quenched with water (20 mL) and concentrated under reduced pressure. The residue was diluted with water (20 mL) and extracted with EtOAc (3 × 50 mL). The organic phase was dried over MgSO₄ and concentrated under reduced pressure. The residue was dissolved in DCM/TFA (1:1, 6 mL) and the mixture was stirred for 48 h under room temperature. The reaction mixture was concentrated under reduced pressure. The residue was dissolved in DCM (50 mL) and washed with 1 M NaOH solution (20 mL). The organic phase was dried, concentrated, and purified by flash chromatography (silica, EtOAc ramping to EtOAc:MeOH = 95:5) to produce the titled compound (1) as a white solid (24 mg, 16%). ¹H NMR (CDCl₃) δ 1.57 (t, 3H, J = 7.0 Hz), 4.35 (q, 2H, J = 7.0 Hz), 5.34 (s, 2H), 6.34 (d, 1H, J = 8.0 Hz), 7.10 (t, 1H, J = 8.5 Hz), 7.25 (m, 5H), 7.35 (m, 2H), 7.49 (d, 1H, J = 8.0 Hz), 7.90 (dd, 1H, J = 1.5 & 12.5 Hz), 8.06 (s, 1H), 11.52 (s, 1H). HRMS m/z 514.1260 [M+H]+.

N-(4-((6-amino-5-chloropyrimidin-4-yl)oxy)-3-fluorophenyl)-1-(4-fluorophenyl)-2-oxo-1,2-dihydropyridine-3-carboxamide (3) was prepared by treating 1-(4-fluorophenyl)-2-oxo-1,2-dihydropyridine-3-carboxylic acid (100 mg, 0.429 mmol) with 6-(4-amino-2-fluorophenoxy)-5-chloro-N,N-di-tert-butoxycarbonylpyrimidin-4-amine (170 mg, 0.374 mmol) as a white solid (132 mg, 75%). ¹H NMR (CDCl₃) δ 5.25 (s, 2H), 6.54 (t, 1H, J = 7.0 Hz), 7.22 (m, 2H), 7.28 (d, 1H, J = 9.5 Hz), 7.34 (m, 2H), 7.55 (d, 1H, J = 6.5 Hz), 7.85 (d, 1H, J = 12.5 Hz), 8.00 (s, 1H), 8.67 (d, 1H, J = 7.5 Hz), 11.88 (s, 1H). HRMS m/z 470.1326 [M+H]⁺.

N-(4-((6-amino-5-chloropyrimidin-4-yl)oxy)phenyl)-1-(4-fluorophenyl)-2-oxo-1,2-dihydropyridine-3-carboxamide (4) was obtained by reacting 1-(4-fluorophenyl)-2-oxo-1,2-dihydropyridine-3-carboxylic acid (100 mg, 0.429 mmol) and 6-(4-aminophenoxy)-5-chloro-N,N-di-tert-butoxycarbonylpyrimidin-4-amine (160 mg, 0.366 mmol) as a white solid (131 mg, 79%). ¹H NMR (CDCl₃) δ 5.30 (s, 2H), 6.60 (t, 1H, J = 7.0 Hz), 7.12 (d, 2H, J = 8.5 Hz), 7.26 (m, 2H), 7.41 (m, 2H), 7.60 (d, 1H, J = 6.5 Hz), 7.79 (d, 2H, J = 8.5 Hz), 8.09 (s, 1H), 8.75 (d, 1H, J = 7.0 Hz), 11.86 (s, 1H). HRMS m/z 452.0980 [M+H]⁺.

N-(4-((6-amino-5-chloropyrimidin-4-yl)oxy)-2-fluorophenyl)-1-(4-fluorophenyl)-2-oxo-1,2-dihydropyridine-3-carboxamide (5) was prepared by treating 1-(4-fluorophenyl)-2-oxo-1,2-dihydropyridine-3-carboxylic acid (100 mg, 0.429 mmol) with 5-chloro-6-(3,4-difluorophenoxy)-N,N-di-tert-butoxycarbonylpyrimidin-4-amine (170 mg, 0.374 mmol) as a light yellow solid (156 mg, 89%). ¹H NMR (CDCl₃) δ 5.34 (s, 2H), 6.59 (t, 1H, J = 7.0 Hz), 6.98 (m, 2H), 7.24 (m, 2H), 7.41 (m, 2H), 7.61 (d, 1H, J = 6.5), 8.10 (s, 1H), 8.60 (m, 1H), 8.73 (d, 1H, J = 7.0 Hz), 12.03 (s, 1H). HRMS m/z 470.0718 [M+H]⁺.

N-(4-((6-amino-5-fluoropyrimidin-4-yl)oxy)phenyl)-1-(4-fluorophenyl)-2-oxo-1,2-dihydropyridine-3-carboxamide (6) was obtained by treating 1-(4-fluorophenyl)-2-oxo-1,2-dihydropyridine-3-carboxylic acid (130 mg, 0.557 mmol) with 6-(4-aminophenoxy)-5-fluoro-N,N- di-tert-butoxycarbonylpyrimidin-4-amine (200 mg, 0.476 mmol) as a beige powder (143 mg, 69%). ¹H NMR (CDCl₃) δ 5.04 (s, 2H), 6.59 (t, 1H, J = 7.0 Hz), 7.13 (d, 2H, J = 9.0 Hz), 7.41 (m, 2H), 7.60 (dd, 1H, J = 1.5 & 6.5 Hz), 7.78 (d, 2H, J = 9.0 Hz), 7.98 (s, 1H), 11.85 (s, 1H) (two proton signals obscured by CDCl₃ peak). HRMS m/z 436.1112 [M+H]⁺.

N-(4-((6-amino-5-fluoropyrimidin-4-yl)oxy)-2-fluorophenyl)-1-(4-fluorophenyl)-2-oxo-1,2-dihydropyridine-3-carboxamide (7) was obtained by treating 1-(4-fluorophenyl)-2-oxo-1,2-dihydropyridine-3-carboxylic acid (100 mg, 0.429 mmol) with 6-(4-amino-3-fluorophenoxy)-5-fluoro-N,N- di-tert-butoxycarbonylpyrimidin-4-amine (160 mg, 0.365 mmol) according to the general synthetic procedure to produce a yellow powder (138 mg, 83%). ¹H NMR (CDCl₃) δ 5.10 (s, 2H), 6.58 (t, 1H, J = 7.0 Hz), 6.99 (m, 2H), 7.24 (m, 2H), 7.41 (m, 2H), 7.60 (d, 1H, J = 6.5 Hz), 7.99 (s, 1H), 8.59 (m, 1H), 8.73 (s, 1H), 12.02 (s, 1H). HRMS m/z 454.1018 [M+H]⁺.

N-(4-((6-amino-5-fluoropyrimidin-4-yl)oxy)-3-fluorophenyl)-2-oxo-1-(4-(trifluoro methoxy)phenyl)-1,2-dihydropyridine-3-carboxamide (8) was prepared by treating 2-oxo-1-(4-(trifluoromethoxy)phenyl)-1,2-dihydropyridine-3-carboxylic acid (78 mg, 0.261 mmol) with 6-(4-amino-2-fluorophenoxy)-5-fluoro-di-tert-butoxycarbonylpyrimidin-4-amine (100 mg, 0.228 mmol) as a white powder (72 mg, 61%). ¹H NMR (DMSO) δ 6.74 (t, 1H, J = 7.0 Hz), 7.33 (m, 3H), 7.42 (d, 1H, J = 9.0 Hz), 7.60 (d, 2H, J = 8.5 Hz), 7.71 (d, 2H, J = 8.5 Hz), 7.82 (s, 1H), 7.93 (d, 1H, J = 12.5 Hz), 8.17 (d, 1H, J = 6.5 Hz), 8.59 (d, 1H, J = 7.0 Hz), 12.01 (s, 1H). HRMS m/z 520.1341 [M+H]⁺.

N-(4-((6-amino-5-fluoropyrimidin-4-yl)oxy)-3-fluorophenyl)-2-oxo-1-(4-(trifluoro methyl)phenyl)-1,2-dihydropyridine-3-carboxamide (9) was prepared by treating 2-oxo-1-(4-(trifluoromethyl)phenyl)-1,2-dihydropyridine-3-carboxylic acid (74 mg, 0.261 mmol) with 6-(4-amino-2-fluorophenoxy)-5-fluoro-di-tert-butoxycarbonylpyrimidin-4-amine (100 mg, 0.228 mmol) as a white powder (85 mg, 74%). ¹H NMR (DMSO) δ 6.78 (t, 1H J = 7.0 Hz), 7.33 (m, 3H), 7.40 (d, 1H, J = 9.0 Hz), 7.81 (m, 3H), 7.92 (d, 1H, J = 12.5 Hz), 7.98 (d, 2H, J = 8.0 Hz), 8.18 (d, 1H, J = 6.5 Hz), 8.60 (d, 1H, J = 7.5 Hz), 11.97 (s, 1H). HRMS m/z 504.1380 [M+H]⁺.

N-(4-((6-amino-5-chloropyrimidin-4-yl)oxy)-3-fluorophenyl)-2-oxo-1-(4-(trifluoro methoxy)phenyl)-1,2-dihydropyridine-3-carboxamide (10) was prepared by treating 2-oxo-1-(4-(trifluoromethoxy)phenyl)-1,2-dihydropyridine-3-carboxylic acid (55 mg, 0.184 mmol) with 6-(4-amino-2-fluorophenoxy)-5-chloro-N,N-di-tert-butoxycarbonylpyrimidin-4-amine (70 mg, 0.160 mmol) as a white powder (37 mg, 43%). ¹H NMR (CDCl₃) δ 5.35 (s, 2H), 6.63 (t, 1H, J = 7.0 Hz), 7.16 (t, 1H, J = 8.5 Hz), 7.34 (d, 1H, J = 8.5 Hz), 7.43 (d, 2H, J = 8.5 Hz), 7.48 (d, 2H, J = 8.5 Hz), 7.61 (d, 1H, J = 6.0 Hz), 7.92 (d, 1H, J = 12.0 Hz), 8.07 (s, 1H), 8.75 (d, 1H, J = 7.0 Hz), 11.90 (s, 1H). HRMS m/z 536.0856 [M+H]⁺.

N-(4-((6-amino-5-chloropyrimidin-4-yl)oxy)-3-fluorophenyl)-2-oxo-1-(4-(trinuoromethyl)phenyl)-1,2-dihydropyridine-3-carboxamide (11) was obtained by treating 2-oxo-1-(4-(trifluoromethyl)phenyl)-1,2-dihydropyridine-3-carboxylic acid (52 mg, 0.184 mmol) with 6-(4-amino-2-fluorophenoxy)-5-chloro-N,N-di-tert-butoxycarbonylpyrimidin-4-amine (70 mg, 0.160 mmol) as a yellow powder (23 mg, 28%). ¹H NMR (DMSO) δ 6.77 (t, 1H, J = 7.0 Hz), 7.32 (t, 1H, J = 8.5 Hz), 7.40 (d, 1H, J = 9.0 Hz), 7.82 (d, 2H, J = 8.0 Hz), 7.95 (m, 4H), 8.18 (d, 1H, J = 6.5 Hz), 8.60 (d, 1H, J = 7.0 Hz), 11.97 (s, 1H). HRMS m/z 520.1093[M+H]⁺.

N-(4-((6-amino-5-chloropyrimidin-4-yl)oxy)-2,3-difluorophenyl)-1-(4-fluorophenyl)-2-oxo-1,2-dihydropyridine-3-carboxamide (13) was prepared by treating 1-(4-fluorophenyl)-2-oxo-1,2-dihydropyridine-3-carboxylic acid (178 mg, 0.763 mmol) with 6-(4-amino-2,3-difluorophenoxy)-5-chloro-N,N-di-tert-butoxycarbonylpyrimidin-4-amine (314 mg, 0.664 mmol) as a white powder (237 mg, 73%). ¹H NMR (DMSO) δ 6.74 (t, 1H, J = 7.0 Hz), 7.25 (t, 1H, J = 8.0 Hz), 7.43 (t, 2H, J = 9.0 Hz), 7.61 (m, 2H), 7.98 (s, 1H), 8.15 (dd, 1H, J = 2.0 & 6.5 Hz), 8.26 (t, 1H, J = 7.5 Hz), 8.61 (dd, 1H, J = 2.0 & 7.5 Hz), 12.31 (s, 1H). MS m/z 488.14

N-(4-((6-amino-5-chloropyrimidin-4-yl)oxy)-2-fluorophenyl)-2-oxo-1-(p-tolyl)-1,2-dihydropyridine-3-carboxamide (15) was prepared by treating 2-oxo-1-(p-tolyl)-1,2-dihydropyridine-3-carboxylic acid (58 mg, 0.253 mmol) with 6-(4-amino-3-fluorophenoxy)-5-chloro-N,N-di-tert-butoxycarbonylpyrimidin-4-amine (100 mg, 0.220 mmol) as a white powder (73 mg, 71%). ¹H NMR (DMSO) δ 2.40 (s, 3H), 6.72 (t, 1H, J = 7.0 Hz), 7.05 (d, 1H, J = 9.0 Hz), 7.29 (dd, 1H, J = 2.0 & 11.5 Hz), 7.38 (m, 4H), 7.98 (s, 1H), 8.10 (dd, 1H, J = 2.0 & 6.5 Hz), 8.47 (t, 1H, J = 9.0 Hz), 8.59 (dd, 1H, J = 2.0 & 7.5 Hz), 12.25 (s, 1H). MS m/z 466.31

N-(4-((6-amino-5-chloropyrimidin-4-yl)thio)-3-fluorophenyl)-1-(4-fluorophenyl)-2-oxo-1,2-dihydropyridine-3-carboxamide (17) was prepared by treating 1-(4-fluorophenyl)-2-oxo-1,2-dihydropyridine-3-carboxylic acid (191 mg, 0.819 mmol) with 6-((4-amino-2-fluorophenyl)thio)-5-chloro-N,N-di-tert-butoxycarbonylpyrimidin-4-amine (335 mg, 0.711 mmol) as a pale yellow powder (67 mg, 19%). ¹H NMR (DMSO) δ 6.73 (t, 1H, J = 7.0 Hz), 7.43 (m, 3H), 7.53 (t, 1H, J = 8.0 Hz), 7.61 (m, 2H), 7.92 (d, 1H, J = 11.0 Hz), 8.00 (s, 1H), 8.14 (dd, 1H, J = 1.5 & 6.5 Hz), 8.58 (dd, 1H, J = 1.5 & 7.0 Hz), 12.19 (s, 1H). MS m/z 486.26.

N-(4-((6-amino-5-fluoropyrimidin-4-yl)thio)-3-fluorophenyl)-1-(4-fluorophenyl)-2-oxo-1,2-dihydropyridine-3-carboxamide (19) was prepared by treating 1-(4-fluorophenyl)-2-oxo-1,2-dihydropyridine-3-carboxylic acid (183 mg, 0.785 mmol) with 6-((4-amino-2-fluorophenyl)thio)-5-fluoro-N,N-di-tert-butoxycarbonylpyrimidin-4-amine (311 mg, 0.684 mmol) according to the general synthetic procedure to produce a beige powder (66 mg, 21%). ¹H NMR (DMSO) δ 6.73 (t, 1H, J = 7.0 Hz), 7.28 (s, 2H), 7.43 (m, 3H), 7.57 (t, 1H, J = 8.5 Hz), 7.61 (m, 2H), 7.92 (m, 2H), 8.14 (dd, 1H, J = 2.0 & 6.5 Hz), 8.59 (dd, 1H, J = 2.0 & 7.5 Hz), 12.19 (s, 1H). MS m/z 470.09.

N-(4-((6-amino-5-fluoropyrimidin-4-yl)oxy)-2,5-difluorophenyl)-1-(4-fluorophenyl)-2-oxo-1,2-dihydropyridine-3-carboxamide (20) was prepared by treating 1-(4-fluorophenyl)-2-oxo-1,2-dihydropyridine-3-carboxylic acid (135 mg, 0.579 mmol) with 6-(4-amino-2,5-difluorophenoxy)-5-fluoro-N,N-di-tert-butoxycarbonylpyrimidin-4-amine (229 mg, 0.502 mmol) according to the general synthetic procedure to produce a white powder (159 mg, 67%). ¹H NMR (DMSO) δ 6.74 (t, 1H, J = 7.0 Hz), 7.37 (s, 2H), 7.43 (t, 1H, J = 8.5 Hz), 7.60 (m, 3H), 7.84 (s, 1H), 8.15 (dd, 1H, J = 2.0 & 6.5 Hz), 8.47 (m, 1H), 8.61 (dd, 1H, J = 2.0 & 7.0 Hz), 12.38 (s, 1H). MS m/z 472.29.

N-(4-((6-amino-5-chloropyrimidin-4-yl)oxy)-2,5-difluorophenyl)-1-(4-fluorophenyl)-2-oxo-1,2-dihydropyridine-3-carboxamide (21) was prepared by treating 1-(4-fluorophenyl)-2-oxo-1,2-dihydropyridine-3-carboxylic acid (122 mg, 0.523 mmol) with 6-(4-amino-2,5-difluorophenoxy)-5-chloro-N,N-di-tert-butoxycarbonylpyrimidin-4-amine (216 mg, 0.457 mmol) according to the general synthetic procedure to produce a white powder (157 mg, 70%). ¹H NMR (DMSO) δ 6.75 (t, 1H, J = 7.0 Hz), 7.43 (t, 2H, J = 9.0 Hz), 7.59 (m, 3H), 7.98 (s, 1H), 8.15 (dd, 1H, J = 2.0 & 6.5 Hz), 8.47 (m, 1H), 8.61 (dd, 1H, J = 2.0 & 7.5 Hz), 12.37 (s, 1H). MS m/z 488.14.

N-(4-((6-amino-5-fluoropyrimidin-4-yl)oxy)-3-chlorophenyl)-1-(4-fluorophenyl)-2-oxo-1,2-dihydropyridine-3-carboxamide (22) was prepared by treating 1-(4-fluorophenyl)-2-oxo-1,2-dihydropyridine-3-carboxylic acid (200 mg, 0.858 mmol) with 6-(4-amino-2-chlorophenoxy)-5-fluoro-N,N-di-tert-butoxycarbonylpyrimidin-4-amine (338 mg, 0.743 mmol) according to the general synthetic procedure to produce a white powder (265 mg, 76%). ¹H NMR (DMSO) δ 6.73 (t, 1H, J = 7.0 Hz), 7.30 (s, 2H), 7.34 (d, 1H, J = 9.0 Hz), 7.42 (t, 2H, J = 8.5 Hz), 7.55 (dd, 1H, J = 2.0 & 8.5 Hz), 7.61 (m, 2H), 7.80 (s, 1H), 8.12 (m, 2H), 8.58 (dd, 1H, J = 1.5 & 7.5 Hz), 12.04 (s, 1H). HRMS m/z 470.15.

N-(4-((6-amino-5-chloropyrimidin-4-yl)oxy)-3-chlorophenyl)-1-(4-fluorophenyl)-2-oxo-1,2-dihydropyridine-3-carboxamide (23) was prepared by treating 1-(4-fluorophenyl)-2-oxo-1,2-dihydropyridine-3-carboxylic acid (154 mg, 0.660 mmol) with 6-(4-amino-2-chlorophenoxy)-5-chloro-N,N-di-tert-butoxycarbonylpyrimidin-4-amine (270 mg, 0.573 mmol) according to the general synthetic procedure to produce a white powder (229 mg, 82%). ¹H NMR (DMSO) δ 6.72 (t, 1H, J = 7.0 Hz), 7.32 (d, 1H, J = 9.0 Hz), 7.42 (t, 2H, J = 8.5 Hz), 7.56 (dd, 1H, J = 2.5 & 9.0 Hz), 7.61 (m, 2H), 7.94 (s, 1H), 8.12 (m, 2H), 8.58 (dd, 1H, J = 2.0 & 7.5 Hz), 12.04 (s, 1H). MS m/z 486.12

N-(4-((2-amino-5-chloropyrimidin-4-yl)oxy)-3-fluorophenyl)-1-(4-fluorophenyl)-2-oxo-1,2-dihydropyridine-3-carboxamide (24) was prepared by treating 1-(4-fluorophenyl)-2-oxo-1,2-dihydropyridine-3-carboxylic acid (78 mg, 0.334 mmol) with 4-(4-amino-2-fluorophenoxy)-5-chloro-N,N-di-tert-butoxycarbonylpyrimidin-2-amine (133 mg, 0.292 mmol) according to the general synthetic procedure to produce a white powder (30 mg, 22%). ¹H NMR (DMSO) δ 6.72 (t, 1H, J = 7.0 Hz), 6.90 (s, 2H), 7.36 (t, 1H, J = 8.5 Hz), 7.42 (m, 3H), 7.60 (m, 2H), 7.94 (dd, 1H, J = 2.0 & 12.5 Hz), 8.13 (dd, 1H, J = 2.0 & 6.5 Hz), 8.58 (dd, 1H, J = 2.0 & 7.5 Hz), 12.34 (s, 1H). MS m/z 470.15.

N-(4-((2-amino-5-chloropyrimidin-4-yl)oxy)-2-fluorophenyl)-1-(4-fluorophenyl)-2-oxo-1,2-dihydropyridine-3-carboxamide (25) was prepared by treating 1-(4-fluorophenyl)-2-oxo-1,2-dihydropyridine-3-carboxylic acid (107 mg, 0.459 mmol) with 4-(4-amino-3-fluorophenoxy)-5-chloro-N,N-di-tert-butoxycarbonylpyrimidin-2-amine (181 mg, 0.398 mmol) according to the general synthetic procedure to produce a yellow powder (73 mg, 39%). ¹H NMR (DMSO) δ 6.73 (t, 1H, J = 7.0 Hz), 6.87 (s, 2H), 7.11 (d, 1H, J = 9.0 Hz), 7.37 (dd, 1H, J = 2.5 & 11.5 Hz), 7.43 (t, 2H, J = 8.5 Hz), 7.61 (m, 2H), 8.13 (dd, 1H, J = 2.0 & 6.5 Hz), 8.21 (s, 1H), 8.46 (t, 1H, J = 9.0 Hz), 8.60 (dd, 1H, J = 2.0 & 7.5 Hz), 12.18 (s, 1H). MS m/z 470.15.

N-(4-((6-amino-5-chloropyrimidin-4-yl)oxy)-2-fluorophenyl)-1-(4-chlorophenyl)-2-oxo-1,2-dihydropyridine-3-carboxamide (33) was prepared by treating 1-(4-chlorophenyl)-2-oxo-1,2-dihydropyridine-3-carboxylic acid (63 mg, 0.252 mmol) with 6-(4-amino-3-fluorophenoxy)-5-chloro-N,N-di-tert-butoxycarbonylpyrimidin-4-amine (100 mg, 0.220 mmol) according to the general synthetic procedure to produce a white powder (68 mg, 64%). ¹H NMR (DMSO) δ 6.74 (t, 1H, J = 7.0 Hz), 7.05 (d, 1H, J = 9.0 Hz), 7.29 (dd, 1H, J = 2.0 & 11.5 Hz), 7.62 (m, 4H), 7.98 (s, 1H), 8.13 (dd, 1H, J = 2.0 & 6.5 Hz), 8.46 (t, 1H, J = 9.0 Hz), 8.60 (dd, 1H, J = 2.0 & 7.0 Hz), 12.17 (s, 1H). MS m/z 486.25

N-(4-((6-amino-5-fluoropyrimidin-4-yl)oxy)-3-fluorophenyl)-1-(4-chlorophenyl)-2-oxo-1,2-dihydropyridine-3-carboxamide (34) was prepared by treating 1-(4-chlorophenyl)-2-oxo-1,2-dihydropyridine-3-carboxylic acid (65 mg, 0.260 mmol) with 6-(4-amino-2-fluorophenoxy)-5-fluoro-N,N-di-tert-butoxycarbonylpyrimidin-4-amine (100 mg, 0.228 mmol) according to the general synthetic procedure to produce a white powder (76 mg, 71%). ¹H NMR (DMSO) δ 6.73 (t, 1H, J = 7.0 Hz), 7.36 (m, 4H), 7.59 (d, 2H, J = 8.5 Hz), 7.66 (d, 2H, J = 8.5 Hz), 7.82 (s, 1H), 7.92 (dd, 1H, J = 2.0 & 12.5 Hz), 8.13 (dd, 1H, J = 2.0 & 6.5 Hz), 8.58 (dd, 1H, J = 2.0 & 7.0 Hz), 12.02 (s, 1H). MS m/z 470.29

N-(4-((6-amino-5-fluoropyrimidin-4-yl)oxy)-3-fluorophenyl)-2-oxo-1-(p-tolyl)-1,2-dihydropyridine-3-carboxamide (35) was prepared by treating 2-oxo-1-(p-tolyl)-1,2-dihydropyridine-3-carboxylic acid (60 mg, 0.262 mmol) with 6-(4-amino-2-fluorophenoxy)-5-fluoro-N,N-di-tert-butoxycarbonylpyrimidin-4-amine (100 mg, 0.228 mmol) according to the general synthetic procedure to produce a white powder (74 mg, 72%). ¹H NMR (DMSO) δ 6.71 (t, 1H, J = 7.0 Hz), 7.35 (m, 8H), 7.82 (s, 1H), 7.92 (dd, 1H, J = 2.0 & 12.5 Hz), 8.09 (dd, 1H, J = 2.0 & 6.5 Hz), 8.57 (dd, 1H, J = 2.0 & 7.5 Hz), 12.12 (s, 1H). MS m/z 450.33

N-(4-((6-amino-5-chloropyrimidin-4-yl)amino)-3-fluorophenyl)-1-(4-fluorophenyl)-2-oxo-1,2-dihydropyridine-3-carboxamide (36) was prepared by treating 1-(4-fluorophenyl)-2-oxo-1,2-dihydropyridine-3-carboxylic acid (67 mg, 0.287 mmol) with N⁴-(4-amino-2-fluorophenyl)-5-chloro-N⁶,N⁶-di-tert-butoxycarbonylpyrimidine-4,6-diamine (113 mg, 0.249 mmol) according to the general synthetic procedure to produce a yellow powder (61 mg, 52%). ¹H NMR (DMSO) δ 6.76 (m, 3H), 7.33 (d, 1H, J = 5.0 Hz), 7.45 (m, 3H), 7.63 (m, 2H), 7.85 (s, 2H), 8.15 (m, 1H), 8.29 (s, 1H), 8.61 (m, 1H), 12.04 (s, 1H). MS m/z 469.10.

N-(4-((6-amino-5-chloropyrimidin-4-yl)oxy)-2-chlorophenyl)-1-(4-fluorophenyl)-2-oxo-1,2-dihydropyridine-3-carboxamide (37) was prepared by treating 1-(4-fluorophenyl)-2-oxo-1,2-dihydropyridine-3-carboxylic acid (167 mg, 0.716 mmol) with 6-(4-amino-3-chlorophenoxy)-5-chloro-N,N-di-tert-butoxycarbonylpyrimidin-4-amine (294 mg, 0.624 mmol) according to the general synthetic procedure to produce an off beige powder (281 mg, 93%). ¹H NMR (DMSO) δ 6.72 (t, 1H, J = 7.0 Hz), 7.21 (dd, 1H, J = 2.5 & 9.0 Hz), 7.43 (m, 3H), 7.61 (m, 2H), 7.98 (s, 1H), 8.12 (dd, 1H, J = 2.0 & 6.5 Hz), 8.57 (d, 1H, J = 9.0 Hz), 8.62 (dd, 1H, J = 2.0 & 7.0 Hz), 12.31 (s, 1H). HRMS m/z 486.0530.

N-(4-((6-amino-5-fluoropyrimidin-4-yl)oxy)-2-chlorophenyl)-1-(4-fluorophenyl)-2-oxo-1,2-dihydropyridine-3-carboxamide (38) was prepared by treating 1-(4-fluorophenyl)-2-oxo-1,2-dihydropyridine-3-carboxylic acid (81 mg, 0.347 mmol) with 6-(4-amino-3-chlorophenoxy)-5-fluoro-N,N-di-tert-butoxycarbonylpyrimidin-4-amine (137 mg, 0.301 mmol) according to the general synthetic procedure to produce a white powder (86 mg, 61%). ¹H NMR (DMSO) δ 6.72 (t, 1H, J = 7.0 Hz), 7.23 (dd, 1H, J = 2.0 & 9.0 Hz), 7.28 (s, 2H), 7.43 (t, 2H, J = 8.5 Hz), 7.48 (d, 1H, J = 2.5 Hz), 7.61 (m, 2H), 7.85 (s, 1H), 8.12 (dd, 1H, J = 1.5 & 7.0 Hz), 8.57 (d, 1H, J = 9.0 Hz), 8.62 (dd, 1H, J = 1.5 & 7.5 Hz), 12.30 (s, 1H). HRMS m/z 470.0826.

N-(4-((6-amino-5-chloropyrimidin-4-yl)thio)-2-fluorophenyl)-1-(4-fluorophenyl)-2-oxo-1,2-dihydropyridine-3-carboxamide (39)

A mixture of 3-fluoro-4-nitrophenol (2.00 g, 12.7 mmol) and DABCO (2.84 g, 25.3 mmol) in anhydrous DMF (10 mL) was treated with dimethylthiocarbamoyl chloride (2.36 g, 19.1 mmol) and the mixture was stirred under nitrogen at 50° C. for 4 h. The reaction mixture was concentrated under reduced pressure. The residue was dissolved in EtOAc (100 mL) and washed with 1 M HCl aqueous solution (50 mL). The organic phase was washed with brine (50 mL), dried, concentrated and purified by flash chromatography (silica, PE ramping to PE:EtOAc = 1:1) to give O-(3-fluoro-4-nitrophenyl) dimethylcarbamothioate as an orange powder (2.172 g, 70%). ¹H NMR (CDCl₃) δ 3.36 (s, 3H), 3.45 (s, 3H), 7.05 (m, 2H), 8.13 (t, 1H, J = 8.5 Hz).

A solution of O-(3-fluoro-4-nitrophenyl) dimethylcarbamothioate (1.00 g, 4.99 mmol) in NMP (10 mL) was heated at 180° C. under microwave irradiation for 20 min. The reaction mixture was concentrated using a Genevac centrifugal evaporator. The residue was purified by flash chromatography (silica, PE ramping to PE:EtOAc = 1:1) to give S-(3-fluoro-4-nitrophenyl) dimethylcarbamothioate as an orange powder (871 mg, 87%). ¹H NMR (CDCl₃) δ 2.83 (s, 6H), 7.40 (d, 1H, J = 8.5 Hz), 7.50 (d, 1H, J = 11.0 Hz), 8.03 (t, 1H, J = 8.0 Hz).

Then, to a solution of S-(3-fluoro-4-nitrophenyl) dimethylcarbamothioate (500 mg, 2.05 mmol) in MeOH/CH₃COOH (1:1, 10 mL) was added iron powder (571 mg, 10.2 mmol). After stirred at 50° C. for 2 h under N₂, iron was removed, and the mixture was concentrated under reduced pressure. The resulting residue was dissolved in DCM (100 mL) and 1 M NaOH solution was added (50 mL). The precipitate was removed by centrifugation. The aqueous layer was extracted with DCM (50 mL). The combined organic layer was dried and concentrated to give S-(4-amino-3-fluorophenyl) dimethylcarbamothioate (419 mg, 96%) as a light-yellow solid which was used for the subsequent reaction without further purification.

A mixture of 1-(4-fluorophenyl)-2-oxo-1,2-dihydropyridine-3-carboxylic acid (524 mg, 2.25 mmol), HATU (892 mg, 2.35 mmol) and DIPEA (0.60 mL, 3.44 mmol) in DCM (10 mL) was stirred for 15 min at room temperature. S-(4-Amino-3-fluorophenyl) dimethylcarbamothioate (419 mg, 1.96 mmol) in DCM (5 mL) was added and the reaction mixture was stirred for 4 h at room temperature. After diluted with DCM (150 mL), saturated NH₄Cl solution (50 mL) was added. The organic phase was separated and dried over MgSO₄ and concentrated under reduced pressure. The residue was purified by flash chromatography (silica gel, PE ramping to EtOAc) to give S-(3-fluoro-4-(1-(4-fluorophenyl)-2-oxo-1,2-dihydropyridine-3-carboxamido)phenyl) dimethylcarbamothioate as a yellow solid (702 mg, 84%). ¹H NMR (CDCl₃) δ 3.05 (m, 6H), 6.58 (t, 1H, J = 7.0 Hz), 7.40 (m, 2H), 7.60 (dd, 1H, J = 2.0 & 6.5 Hz), 8.60 (t, 1H, J = 8.0 Hz), 8.72 (dd, 1H, J = 2.0 & 7.5 Hz), 12.13 (s, 1H) (one proton signal obscured by CDCl₃ peak).

A mixture of S-(3-fluoro-4-(1-(4-fluorophenyl)-2-oxo-1,2-dihydropyridine-3-carboxamido)phenyl) dimethylcarbamothioate (700 mg, 1.63 mmol) in THF/MeOH/H₂O (2:2:1, 25 mL) was treated with lithium hydroxide (80 mg, 3.34 mmol) and the reaction mixture was stirred at 80° C. for 15 h. After concentrated, the residue was added with 1 M HCl aqueous solution and extracted with EtOAc (3 × 50 mL). The extracts were combined, washed with brine (50 mL) and concentrated to give N-(2-fluoro-4-mercaptophenyl)-1-(4-fluorophenyl)-2-oxo-1,2-dihydropyridine-3-carboxamide as a yellow powder (510 mg, 87%). ¹H NMR (DMSO) δ 5.68 (s, 1H), 6.72 (t, 1H, J = 7.0 Hz), 7.13 (d, 1H, J = 8.5 Hz), 7.29 (dd, 1H, J = 1.5 & 11.5 Hz), 7.42 (m, 3H), 7.59 (m, 3H), 8.12 (dd, 1H, J = 2.0 & 6.5 Hz), 8.34 (t, 1H, J = 8.5 Hz), 8.57 (dd, 1H, J = 2.0 & 7.5 Hz), 12.15 (s. 1H).

Finally, a mixture of N-(2-fluoro-4-mercaptophenyl)-1-(4-fluorophenyl)-2-oxo-1,2-dihydropyridine-3-carboxamide (216 mg, 0.603 mmol), 5,6-dichloro-N,N-di-tert-butoxycarbonylpyrimidin-4-amine (200 mg, 0.549 mmol) and caesium carbonate (215 mg, 0.660 mmol) in DMF (5 mL) was stirred at room temperature for 12 h. After concentrated, the residue was dissolved in DCM (100 mL) and washed with water (25 mL). The organic phase was dried over MgSO₄ and concentrated under reduced pressure. The residue was purified by flash chromatography (silica, PE ramping to PE:EtOAc = 1:4). The resulting product was treated with TFA in CH₂Cl₂ (1:1, 6 mL) for 4 h at room temperature. After concentrated, the residue was dissolved in DCM (50 mL) and washed with 1 M NaOH solution (20 mL). The organic phase was dried over MgSO₄ and concentrated to give N-(4-((6-amino-5-chloropyrimidin-4-yl)thio)-2-fluorophenyl)-1-(4-fluorophenyl)-2-oxo-1,2-dihydropyridine-3-carboxamide (48) as a beige powder (134 mg, 50%). ¹H NMR (DMSO) δ 6.74 (t, 1H, J = 7.0 Hz), 7.41 (m, 3H), 7.54 (dd, 1H, J = 2.0 & 11.0 Hz), 7.61 (m, 2H), 8.03 (s, 1H), 8.15 (dd, 1H, J = 2.0 & 6.5 Hz), 8.56 (t, 1H, J = 8.5 Hz), 8.62 (dd, 1H, J = 2.0 & 7.5 Hz), 12.38 (d, 1H, J = 2.0 Hz). HRMS m/z 486.0598.

### Example 2 Biological Activity

### Kinase Assays

Filter-binding radiometric kinase activity assay (Kinase Profiler™) from Eurofins Discovery was used to determine the % of inhibition and/or IC₅₀ values. Briefly, optimised concentrations of TYRO3, AXL, MER and MET human kinases are incubated with 8 mM MOPS pH 7.0, 0.2 mM EDTA, 250 µM of specific substrates (ie TYRO3: KVEKIGEGTYGVVYK (SEQ ID NO: 1); AXL: KKSRGDYMTMQIG (SEQ ID NO: 2); MER: GGMEDIYFEFMGGKKK (SEQ ID NO: 3) and Met: KKKGQEEEYVFIE (SEQ ID NO: 4), respectively), 10 mM Magnesium acetate, [γ″P]-ATP within 15 µM of the apparent Kₘ for ATP (AXL/MET: 90 µM and Mer/TYRO3 45 µM) and test compounds. The reaction is initiated by the addition of the Mg/ATP mixture. After incubation for 40 minutes at room temperature, the reaction is stopped by the addition of phosphoric acid to a concentration of 0.5%. 10 µL of the reaction is then spotted onto a P30 filter mat and washed four times for 4 minutes in 0.425% phosphoric acid and once in methanol prior to drying and scintillation counting. The IC₅₀ values were derived by fitting a sigmoidal dose-response curve to a plot of assay readout over inhibitor concentration. All fits were computed with the GraphPad Prism Software (San Diego, CA, United States of America). Kᵢ values were derived from IC₅ₒ values using Cheng Prusoff equation (Cheng Y et al., Biochem Pharmacol 22(23):3099-3108, 1973).

Inhibition of CDKs and FLT3 were determined using ADP Glo Kinase assays, as previously described in International Patent Publication No WO 2017/020065. The results are shown in Table 2.

### Proliferation Assay

Compounds from Example 1 were subjected to standard resazurin and MTT assays on solid tumour and leukaemia cancer cell lines, respectively, as previously reported (Wang S et al., J Med Chem 47:1662-1675, 2004 and Diab S et al., CheMedChem 9:962-972, 2014). The results are shown in Table 2.

TABLE 2

Enzymatic and cellular activity of example compounds

| Cmpd | % Residual enzymatic activity at 1 µM | | | | | | | Proliferation | |
|---|---|---|---|---|---|---|---|---|---|
| | | | | | | | | % at 1 µM | GI₅₀ (µM) |
| | AXL | Mer | TYRO3 | MET | CDK2E | CDK4D1 | FLT3-ITD | MV4-11 | MDA-MB-231 |
| 1 | 13 | 12 | -1 | 0 | 99 | 40 | 50 | 3 | 1.2 |
| 2 | 2 | 1 | 0 | 2 | 94 | 38 | 52 | 15 | - |
| 3 | 4 | 6 | 0 | -7 | 96 | 69 | 49 | 3 | 7.9 |
| 4 | -1 | 19 | 2 | -5 | 97 | 48 | 33 | 6 | 2.7 |
| 5 | -8 | 0 | 2 | -5 | 92 | 72 | 43 | 6 | 0.9 |
| 6 | 14 | 14 | 6 | 8 | 89 | 69 | 79 | 3 | 8.9 |
| 7 | 8 | 4 | 5 | -6 | 80 | 72 | 40 | 6 | 2.3 |
| 8 | 112 | 100 | 111 | 85 | - | - | 91 | - | - |
| 9 | 105 | 85 | 106 | 64 | - | - | 88 | - | - |
| 13 | 67 | 91 | 87 | 94 | - | - | 74 | 7 | - |
| 15 | 64 | 99 | 39 | 74 | - | - | - | 26 | - |
| 17 | 55 | 81 | 52 | 65 | 101 | - | - | 4 | - |
| 19 | 84 | 99 | 89 | 78 | 93 | - | - | 6 | - |
| 20 | 31 | 54 | 2 | 4 | 93 | - | - | 5 | - |
| 21 | 3 | 30 | 3 | 5 | - | - | 22 | 3 | - |
| 22 | 57 | 89 | 63 | 93 | - | - | 43 | 3 | - |
| 23 | 67 | 95 | 63 | 98 | - | - | 51 | 1 | - |
| 24 | 99 | 87 | 94 | 74 | - | - | - | 27 | - |
| 25 | 92 | 88 | 89 | 87 | - | - | - | 65 | - |
| 33 | 23 | 74 | 6 | 10 | 82 | - | - | 1 | - |
| 34 | 50 | 103 | 69 | 91 | 97 | - | - | 3 | - |
| 35 | 70 | 99 | 14 | 60 | 97 | - | - | 6 | - |
| 36 | 97 | 99 | 70 | 87 | 96 | - | - | 4 | - |
| 37 | 78 | 87 | 39 | 88 | 96 | - | - | 48 | - |
| 38 | 69 | 92 | 50 | 68 | 90 | - | - | 43 | - |
| 39 | 87 | 82 | - | 70 | 88 | - | - | 4 | |

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the present disclosure is not restricted in its use to the particular application described. Neither is the present disclosure restricted in its preferred embodiment with regard to the particular elements and/or features described or depicted herein. It will be also appreciated that the disclosure is not limited to the embodiment or embodiments disclosed, but is capable of numerous rearrangements, modifications and substitutions without departing from the scope of the disclosure as set forth and defined by the following claims.

1. A compound of Formula I: 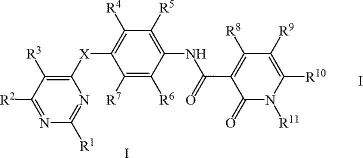
wherein:
X is O or S;
R¹, R², R³, R⁴, R⁵, R⁶, R⁷, R⁸, R⁹ and R¹⁰ are each independently selected from the group consisting of H, alkyl, alkyl-R¹², aryl, aryl-R¹², aralkyl, aralkyl-R¹², alicyclic, heterocyclic, halogen, NO₂, CN, CF₃, O—CF₃, OH, O-alkyl, COR¹², COOR¹², O-aryl, O-R¹², amino, NH-alkyl, NH-aryl, N-(alkyl)₂, N-(aryl)₂, N-(alkyl)(aryl), NH-R¹², NH-alkyl-N(alkyl)₂, N-(R¹²)(R¹³), N-(alkyl)(R¹²), N-(aryl)(R¹²), COOH, CONH₂, CONH-alkyl, CONH-aryl, CONH-alicyclic, CON-(alkyl)(R¹²), CON(aryl)(R¹²), CONH—R¹², CON-(R¹²)(R¹³), S-alkyl, SO₃H, SO₂-alkyl, SO₂-alkyl-R¹², SO₂-aryl, SO₂-aryl-R¹², SO₂NH₂, SO₂NH—R¹², SO₂N—(R¹²)(R¹³), CO-alkyl, CO-alkyl-R¹², CO-aryl and CO-aryl-R¹², wherein said alkyl, aryl, aralkyl, alicyclic and heterocyclic groups may be optionally substituted with one or more groups selected from C₁₋₆ alkyl, O-C₁₋₆ alkyl, CN, OH, NH₂, COOH, CONH₂, CF₃, OCF₃ and halogen; wherein R¹² and R¹³ are independently selected from COOH, SO₃H, OSO₃H, SONHCH₃, SONHCH₂CH₃, SO₂CH₃, SO₂CH₂CH₃, PO₃H₂ and OPO₃H₂, mono-, di- and poly-hydroxylated alicyclic groups, di- or poly-hydroxylated aliphatic or aryl groups, and N-, O- and/or S-containing heterocyclic groups optionally substituted with one or more C₁₋₆ alkyl, hydroxyl, carbonyl, amino or alkoxy groups, wherein the N-, O- and/or S-containing heterocyclic groups may optionally be linked to the rest of the compound through an alkyl, amine, alkoxy or ketone bridge, wherein at least two of R¹, R² and R³ are other than H; and
R¹¹ is selected from phenyl-R¹⁴,
wherein R¹⁴ is selected from C₁₋₆ alkyl, O-C₁₋₆ alkyl, CN, OH, NH₂, COOH, CONH₂, CF₃, OCF₃ and halogen;
or a pharmaceutically acceptable salt, solvate or prodrug thereof.

2. The compound according to claim 1, wherein R₁, R₂ and R₃ are independently selected from the group consisting of H, C₁₋₆ alkyl, CN, CF₃, NH₂, O-C₁₋₆ alkyl, NH-C₁₋₆ alkyl, S-C₁₋₆ alkyl, and halogen.

3. The compound according to claim 2, wherein R₁ is H, C₁₋₃ alkyl or NH₂.

4. The compound according to claim 1, wherein R₂ is H, C₁₋₃ alkyl or NH₂.

5. The compound according to claim 1, wherein R₃ is H, C₁₋₃ alkyl, O-alkyl or halogen.

6. The compound according to claim 1, wherein R⁴, R⁵, R⁶ and R⁷ are independently selected from the group consisting of H, C₁₋₆ alkyl, CN, CF₃, NH₂, O-C₁₋₆ alkyl, NH-C₁₋₆ alkyl, S-C₁₋₆ alkyl, and halogen.

7. The compound according to claim 6, wherein R⁴, R⁵, R⁶ and R⁷ are independently selected from H and halogen.

8. The compound according to claim 1, wherein at least one of R⁴, R⁵, R⁶ and R⁷ is H.

9. The compound according to claim 1, wherein one or two of R⁴, R⁵, R⁶ and R⁷ are halogen.

10. The compound according to claim 6, wherein R⁴, R⁵, R⁶ and R⁷ are all H.

11. The compound according to claim 1, wherein R⁵, R⁹ and R¹⁰ are independently selected from the group consisting of H, C₁₋₆ alkyl, CN, CF₃, NH₂, O-C₁₋₆ alkyl, NH-C₁₋₆ alkyl, S-C₁₋₆ alkyl, and halogen.

12. The compound according to claim 1, wherein R₅ is H, C₁₋₃ alkyl or O-C₁₋₃ alkyl.

13. The compound according to claim 1, wherein at least one of R⁹ and R¹⁰ is H.

14. The compound according to claim 1, wherein R¹¹ is selected from phenyl-R¹⁴, wherein R¹⁴ is selected from C₁₋₃ alkyl, O-C₁₋₃ alkyl, CF₃, OCF₃ and halogen.

15. The compound according to claim 14, wherein R¹¹ is phenyl-R¹⁴, wherein R¹⁴ is selected from CH₃, OCH₃, CF₃, OCF₃, F and Cl.

16. The compound according to claim 15, wherein R¹¹ is fluorophenyl.

17. The compound of claim 1 selected from the group consisting of:
N-(4-((6-amino-5-fluoropyrimidin-4-yl)oxy)-3-fluorophenyl)-1-(4-fluorophenyl)-2-oxo-1,2-dihydropyridine-3-carboxamide
N-(4-((6-amino-5-chloropyrimidin-4-yl)oxy)-3-fluorophenyl)-4-ethoxy-1-(4-fluorophenyl)-2-oxo-1,2-dihydropyridine-3-carboxamide
N-( 4-(( 6-amino-5-chloropyrimidin-4-yl)oxy)-3-fluorophenyl)-l-( 4-fluorophenyl)-2-oxo-1,2-dihydropyridine-3-carboxamide
N-(4-((6-amino-5-chloropyrimidin-4-yl)oxy)phenyl)-1-(4-fluorophenyl)-2-oxo-1,2-dihydropyridine-3-carboxamide
N-(4-((6-amino-5-chloropyrimidin-4-yl)oxy)-2-fluorophenyl)-1-(4-fluorophenyl)-2-oxo-1,2-dihydropyridine-3-carboxamide
N-(4-((6-amino-5-fluoropyrimidin-4-yl)oxy)phenyl)-1-(4-fluorophenyl)-2-oxo-1,2-dihydropyridine-3-carboxamide
N-(4-((6-amino-5-fluoropyrimidin-4-yl)oxy)-2-fluorophenyl)-1-(4-fluorophenyl)-2-oxo-1,2-dihydropyridine-3-carboxamide
N-(4-((6-amino-5-fluoropyrimidin-4-yl)oxy)-2,3-difluorophenyl)-1-(4-fluorophenyl)-2-oxo-1,2-dihydropyridine-3-carboxamide
N-(4-((6-amino-5-chloropyrimidin-4-yl)oxy)-2,3-difluorophenyl)-1-(4-fluorophenyl)-2-oxo-1,2-dihydropyridine-3-carboxamide
N-(4-((6-amino-5-chloropyrimidin-4-yl)oxy)-2-fluorophenyl)-2-oxo-1-(p-tolyl)-1,2-dihydropyridine-3-carboxamide
N-(4-((6-amino-5-fluoropyrimidin-4-yl)oxy)-2,5-difluorophenyl)-1-(4-fluorophenyl)-2-oxo-1,2-dihydropyridine-3-carboxamide
N-(4-((6-amino-5-chloropyrimidin-4-yl)oxy)-2,5-difluorophenyl)-1-(4-fluorophenyl)-2-oxo-1,2-dihydropyridine-3-carboxamide
N-(4-((6-amino-5-fluoropyrimidin-4-yl)oxy)-3-chlorophenyl)-1-(4-fluorophenyl)-2-oxo-1,2-dihydropyridine-3-carboxamide
N-(4-((6-amino-5-chloropyrimidin-4-yl)oxy)-3-chlorophenyl)-1-(4-fluorophenyl)-2-oxo-1,2-dihydropyridine-3-carboxamide
N-(4-((2-amino-5-chloropyrimidin-4-yl)oxy)-3-fluorophenyl)-1-(4-fluorophenyl)-2-oxo-1,2-dihydropyridine-3-carboxamide
N-(4-((6-amino-5-fluoropyrimidin-4-yl)oxy)-3-fluorophenyl)-4-ethoxy-1-(4-fluorophenyl)-2-oxo-1,2-dihydropyridine-3-carboxamide
N-(4-((6-amino-5-fluoropyrimidin-4-yl)oxy)-2-fluorophenyl)-4-ethoxy-1-(4-fluorophenyl)-2-oxo-1,2-dihydropyridine-3-carboxamide
N-(4-((6-amino-5-chloropyrimidin-4-yl)oxy)-2-fluorophenyl)-4-ethoxy-1-(4-fluorophenyl)-2-oxo-1,2-dihydropyridine-3-carboxamide
N-(4-((6-amino-5-chloropyrimidin-4-yl)oxy)-2-fluorophenyl)-1-(4-chlorophenyl)-2-oxo-1,2-dihydropyridine-3-carboxamide
N-(4-((6-amino-5-fluoropyrimidin-4-yl)oxy)-3-fluorophenyl)-1-(4-chlorophenyl)-2-oxo-1,2-dihydropyridine-3-carboxamide
N-(4-((6-amino-5-fluoropyrimidin-4-yl)oxy)-3-fluorophenyl)-2-oxo-1-(p-tolyl)-1,2-dihydropyridine-3-carboxamide
N-(4-((6-amino-5-chloropyrimidin-4-yl)amino)-3-fluorophenyl)-1-(4-fluorophenyl)-2-oxo-1,2-dihydropyridine-3-carboxamide
N-(4-((6-amino-5-chloropyrimidin-4-yl)thio)-2-fluorophenyl)-1-(4-fluorophenyl)-2-oxo-1,2-dihydropyridine-3-carboxamide.

18. (canceled)

19. A method of treating cancer or another proliferative cell disease or condition in a subject, the method comprising administering to said subject a therapeutically effective amount of a compound according to claim 1 or a pharmaceutically acceptable salt, solvate or prodrug thereof, optionally in combination with a pharmaceutically acceptable carrier, diluent and/or excipient.

20. (canceled)

21. A pharmaceutical composition or medicament comprising a compound according to claim 1, or a pharmaceutically acceptable salt, solvate or prodrug thereof, and a pharmaceutically acceptable carrier, diluent and/or excipient.

22. A method for modulating protein kinase activity in a cell, comprising introducing to or contacting said cell with an effective amount of a compound according to claim 1 or a pharmaceutically acceptable salt, solvate or prodrug thereof.

23. Themethod according to claim 22, wherein the method modulates the activity of one or more protein kinase selected from RTKs.
